(19) United States
(12) Patent Application Publication
(10) Pub. No.: US 2025/0195691 A1
(43) Pub. Date: Jun. 19, 2025

(54) GENE AUGMENTATION THERAPIES FOR INHERITED RETINAL DEGENERATION CAUSED BY MUTATIONS IN THE PRPF31 GENE

(71) Applicant: Massachusetts Eye and Ear Infirmary, Boston, MA (US)

(72) Inventors: Eric A. Pierce, Belmont, MA (US); Michael H. Farkas, Revere, MA (US); Maria E. Sousa, Dedham, MA (US)

(21) Appl. No.: 18/927,466

(22) Filed: Oct. 25, 2024

Related U.S. Application Data
(63) Continuation of application No. 16/992,689, filed on Aug. 13, 2020, now abandoned.
Application No. 16/992,689 is a continuation of application No. 15/555,915, filed on Sep. 5, 2017, now abandoned.
(60) Provisional application No. 62/147,307, filed on Apr. 14, 2015.
(60) Provisional application No. 62/129,638, filed on Mar. 6, 2015.

(51) Int. Cl. A61K 48/00; A61K 9/00; C12N 15/113; C12N 15/63; C12N 15/90

(52) U.S. Cl. CPC A61K 48/005; A61K 9/0048; C12N 15/63; C12N 15/90; C12N 15/113; C12N 2310/20; C12N 2750/14143; C12N 2800/22; C12N 2830/008

(57) ABSTRACT

The present invention relates to methods and compositions for gene therapy of retinitis pigmentosa related to mutations in pre-mRNA processing factor 31 (PRPF31).

### CROSS-REFERENCE TO RELATED APPLICATIONS

This application is a continuation of U.S. patent application Ser. No. 16/992,689, filed Aug. 13, 2020, which is a continuation of U.S. patent application Ser. No. 15/555,915, filed Sep. 5, 2017, now abandoned, which is a § 371 U.S. National Phase Application of International Application No. PCT/US2016/021226, filed on Mar. 7, 2017, and claims the benefit of U.S. Application Ser. Nos. 62/147,307, filed on Apr. 14, 2015, and 62/129,638, filed on Mar. 6, 2015. The entire contents of the foregoing are incorporated herein by reference.

### FEDERALLY SPONSORED RESEARCH OR DEVELOPMENT

This invention was made with Government support under Grant No. EY020902 awarded by the National Institutes of Health. The Government has certain rights in the invention.

### SEQUENCE LISTING

This application contains a Sequence Listing that has been submitted electronically as an XML file named “00633-0192003_SL_ST26.XML.” The XML file, created on Oct. 24, 2024, is 131,846 bytes in size. The material in the XML file is hereby incorporated by reference in its entirety.

### TECHNICAL FIELD

The present invention relates to methods and compositions for gene therapy of retinitis pigmentosa related to mutations in pre-mRNA processing factor 31 (PRPF31).

### BACKGROUND

Mutations in the Pre-mRNA Processing Factor 31 (PRPF31) cause non-syndromic retinitis pigmentosa (RP) in humans, an inherited retinal dystrophy (IRD). It is currently unclear what mechanisms, or which tissues, are affected when mutations are present in these ubiquitously expressed proteins.

### SUMMARY

Described herein are methods and compositions for gene therapy of retinitis pigmentosa related to mutations in pre-mRNA processing factor 31 (PRPF31).

Thus, provided herein are methods for treating retinitis pigmentosa caused by mutations in PRPF31 in a human subject, or for increasing expression of PRPF31 in the eye of a human subject. The methods include delivering to the eye of the subject a therapeutically effective amount of an adeno-associated viral vector, e.g., an Adeno-associated virus type 2 (AAV2) vector, comprising a sequence encoding human PRPF31, operably linked to a promoter that drives expression in retinal pigment epithelial (RPE) cells.

The promoter can be RPE-specific or can be a general promoter that drives expression in other cells types as well, e.g., CASI or CAG. In some embodiments, the promoter is an RPE65 or VMD2 promotor.

In some embodiments, the PRPF31 sequence is codon optimized, e.g., for expression in human cells where the subject is a human. In some embodiments, the PRPF31 sequence is or comprises, or encodes the same protein as, nts 1319-2818 of SEQ ID NO:34.

In some embodiments, the vector is delivered via sub-retinal injection.

In some embodiments, the vector comprises, or comprises a sequence encoding, an AAV capsid polypeptide described in WO 2015054653.

Also provided herein are adeno-associated viral vectors, e.g., adeno-associated virus type 2 (AAV2) vectors comprising a sequence encoding human PRPF31, operably linked to a promotor that drives expression in retinal pigment epithelial (RPE) cells. The promoter can be RPE-specific or can be a general promoter that drives expression in other cells types as well, e.g., CASI or CAG. In some embodiments, the promotor is an RPE65 or VMD2 promotor. In some embodiments, the PRPF31 sequence is codon optimized, e.g., for expression in human cells. Also provided are pharmaceutical compositions comprising the vector, preferably formulated for delivery via sub-retinal injection.

Also provided herein is the use of the nucleic acids, vectors, and pharmaceutical compositions described herein

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-F. Inhibition of phagocytosis in Prpf-mutant mice. Retinal pigment epithelial (RPE) primary cultures were established from 9-10-day old Prpf-mutant mice and their littermate controls, then challenged with FITC-labeled porcine photoreceptor outer segments and nuclei labeled with DAPI (blue). (A) Qualitative representation of primary RPE cells (blue DAPI staining of nuclei) from wild-type (WT) or Prpf3T494M/T494M, Prpf8H2309P/H2309P, Prpf31+/−mutant (MUT) mice. A difference in POS uptake was observed between the mutants and controls. (B) Quantitative analysis of the phagocytic ratio demonstrates a significant decrease in phagocytosis in the mutant (MUT) mice compared to wild-type littermates (WT) for the 3 mutant mouse strains as indicated (*P<0.05, N=3-5). (C) Binding and internalization ratios of POS was compared between Prpf31+/−(MUT) mice compared to wild-type controls (WT) showing a significant decrease in binding (Bind.), but no significant change in POS internalization (Intern.) in mutant mice (*P<0.05, N=2-5). (D) A stable line of shRNA-mediated knockdown of PRPF31 in ARPE-19 cells. A difference in POS uptake was also observed between the control shRNA-transfected ARPE-19 cells and anti-PRPF31 shRNA-transfected ARPE-19cells. (E) Cell viability assay to determine the effect of PRPF31-knockdown in ARPE-19 cells shows that there are no significant differences in cell growth or viability following shRNA-knockdown of PRPF31 relative to the non-targeted control (P>0.05, N=6). (F) shRNA-mediated knockdown of PRPF31 in the human ARPE-19 cell line also inhibits phagocytosis significantly as showed by the decreased number of POS per cell compared to non-targeting constructs (NTC) (*P<0.05, N=3). Error bars represent standard deviation from the mean.

FIGS. 2A-C. The diurnal rhythmicity of phagocytosis in Prpf-mutant mice is disrupted. Phagocytosis was assayed in vivo at 2 hours before light onset (−2), at light onset (0), and 2, 4, and 6 (+2, +4, +6) hours after light onset. (A, B) Representative pictures are shown at +2 (phagocytic peak) and +8 (outside of the phagocytic peak) hours after light onset as indicated. RPE: retinal pigment epithelium, OS: photoreceptor outer segments, Ch: choroid. (A) Detection of early phagosomes in Prpf3- and Prpf8-mutant mice was performed using electron microscopy and counting phagosomes that were 1) in the cytoplasm of the RPE and 2) contained visible lamellar structure (black arrowheads). Scale bar 2 μm. (B) The diurnal rhythm of Prpf31+/−mice was determined using immunofluorescent staining for rhodopsin (Ig-AlexaFluor488) and detection of phagosomes (white arrowheads) located in the RPE cell layer (DAPI-stained nuclei) across 100 μm of intact retina. Scale bar 20 μm. (C) Phagosome quantification across all time points demonstrates the consistent significant disruption of the phagocytic burst in all Prpf-mutant mice (*P<0.05, N=2 for Prpf3- and Prpf8-mutant and N=3-5 for Prpf31-mutant mice). Error bars represent standard deviation from the mean.

FIGS. 3A-B. Alterations in retinal adhesion in Prpf-mutant mice at the peak time-point. Adhesive strength between RPE apical microvilli and POS was determined by quantifying the amount of RPE pigments or proteins that adheres to the neural retina, relative to the WT control. (A) Melanin quantification demonstrates that adhesion is decreased in all three mutant mice at the peak time-point 3.5 hours after light onset, and in Prpf8H2309P/H2309P mice at the off-peak time-point (*P<0.05, N=3-7). (B) Quantitative measurements of RPE65 proteins on immunoblots confirm the melanin findings in all three mutant mice at the peak time-point, however only a trend is observed for decrease in adhesion at the off-peak time-point in Prpf8-mutant mice (*P<0.05, N=4-7). Error bars represent standard deviation from the mean.

FIGS. 4A-C. Localization and expression of some adhesion and phagocytosis markers are perturbed in Prpf3- and Prpf8-mutant mice. Representative images of the expression and localization of (A) αv and β5 integrin receptor subunits and associated Mfg-E8 ligand, (B) FAK intracellular signaling protein, and (C) MerTK receptors and associated Gas6 and Protein S ligands on wild-type control (WT) as well as Prpf3- and Prpf8-mutant retinal cryosections as indicated. Images from sections probed with non-immune IgG (IgG) are included for each antigen. RPE: retinal pigment epithelium, OS: photoreceptor outer segments, ONL: outer nuclear layer. Localization of β5 integrin to the basal side of the RPE was observed in both Prpf3- and Prpf8-mutant mice. Additionally, FAK was mislocalized in Prpf8-mutant mice to the basal side of the RPE. Each protein of interest was stained with Ig-AlexaFluor488 and nuclei are stained with DAPI. Scale bar 40 μm.

FIGS. 5A-C. Localization and expression of some adhesion and phagocytosis markers are perturbed in Prpf31-mutant mice. Representative images of the expression and localization of (A) αv and β5 integrin receptor subunits and associated Mfg-E8 ligand, (B) FAK intracellular signaling protein, and (C) MerTK receptors and associated Gas6 and Protein S ligands or non-immune IgG (IgG) on wild-type control (WT) as well as Prpf31-mutant retinal paraffin sections as indicated. RPE: retinal pigment epithelium, OS: photoreceptor outer segments, ONL: outer nuclear layer. The most notable change in Prpf31-mutant mice is the mislocalization of β5 integrin to the basal side of the RPE, while localization of MerTK is also perturbed. Each protein of interest was stained with IgG-AlexaFluor488 and nuclei are stained with DAPI. Scale bar 20 μm.

FIG. 6. Sequence of PRPF31^{+/-} hiPSC and ARPE-19 cell lines generated via genome editing. The gene model for PRPF31 is shown above, with sequence detail in exons 6-7 for the three example cell lines shown below. The knockout hiPSC cell line has a heterozygous 4 bp deletion (deleted bases shown overlined in normal sequence), which results in a frame shift (underlined amino acids), and premature stop. The knockout ARPE-19 cell lines depicted have a 4 bp deletion or a single base insertion which also result in frameshifts and null alleles.

FIG. 7. Relative POS uptake following treatment with AAV.CASI.PRPF31. Genome-edited PRPF31 (GE31) ARPE-19 cells were transduced with AAV.CASI.PRPF31 at MOIs of 0, 10,000, and 15,000 for 3 days. Subsequently, the cells were incubated with FITC-POS for 1 hour and FITC positive cells were counted by flow cytometry. *P<0.05.

### DETAILED DESCRIPTION

Mutations in genes that encode RNA splicing factors are the second most common cause of the dominant form of the blinding disorder retinitis pigmentosa (RP), and thus are an important cause of vision loss (Hartong et al., Lancet. 2006; 368:1795-809; Daiger et al., Archives Ophthalmology. 2007; 125:151-8; Sullivan et al., Investigative Ophthalmology & Visual Science. 2013; 54:6255-61. PMCID: 3778873). The splicing factors affected, pre-mRNA processing factor (PRPF) 3, PRPF4, PRPF6, PRPF8, PRPF31, and SNRNP200 are highly conserved components of the spliceosome, the complex which excises introns from nascent RNA transcripts to generate mature mRNAs (McKie et al., Human Molecular Genetics. 2001; 10:1555-62; Vithana et al., Molecular Cell. 2001; 8:375-81; Chakarova et al., Human Molecular Genetics. 2002; 11:87-92; Keen et al., European Journal Human Genetics. 2002; 10:245-9; Nilsen, Bioessays. 2003; 25:1147-9; Sullivan et al., Investigative Ophthalmology & Visual Science. 2006; 47:4579-88; Zhao et al., American Journal Human Genetics. 2009; 85:617-27; Tanackovic et al., American Journal Human Genetics. 2011; 88:643-9; Chen et al., Human Molecular Genetics. 2014; 23:2926-39.). Mutations in the PRPF31 gene are the most common cause of RNA splicing factor RP, and are estimated to account for 2400 to 8500 affected individuals in the US and 55,000 to 193,000 people worldwide (Daiger et al., Archives Ophthalmology. 2007; 125:151-8; Sullivan et al., Investigative Ophthalmology & Visual Science. 2013; 54:6255-61. PMCID: 3778873). Since RNA splicing is required in all cells, it is not clear how mutations in these ubiquitous proteins lead to retina-specific disease.

To understand the mechanism(s) by which mutations in RNA splicing factors cause retinal degeneration, the phenotypes of Prpf3, Prpf8 and Prpf31 mutant mice were studied. Cell autonomous defects were identified in retinal pigment epithelial (RPE) cell function in gene targeted mice; however, genetic and phenotypic differences in disease between the mouse models and the human condition make conclusions drawn in mice potentially difficult to translate to humans.

There is some evidence that mutations in PRPF31 cause disease via haploinsuffiency, and thus that this form of dominant RP is amenable to treatment with gene augmentation therapy. Many of the mutations identified in PRPF31 are either large chromosomal deletions or are nonsense and frameshift mutations that lead to premature termination codons that undergo nonsense mediated mRNA decay and result in null alleles (Vithana et al., Molecular Cell. 2001; 8:375-81; Sullivan et al., Investigative Ophthalmology & Visual Science. 2006; 47:4579-88.; Wang et al., American Journal Medical Genetics A. 2003; 121A: 235-9; Xia et al., Molecular Vision. 2004; 10:361-5; Sato et al., American Journal Ophthalmology. 2005; 140:537-40; Abu-Safieh et al., MolVis. 2006; 12:384-8; Rivolta et al., Human Mutation. 2006; 27:644-53; Waseem et al., Investigative Ophthalmology & Visual Science. 2007; 48:1330-4; Rio Frio et al., Human Mutation. 2009; 30:1340-7. PMCID: 2753193; Rose et al., Investigative Ophthalmology & Visual Science. 2011; 52:6597-603; Saini et al., Experimental Eye Research. 2012; 104:82-8). Thus, it is thought that PRPF31-associated retinal degeneration is caused by haploinsufficiency. Consistent with this hypothesis, the level of PRPF31 expression from the wild-type allele correlates with the severity of disease in patients with mutations in PRPF31 (Rio et al., Journal Clinical Investigation. 2008; 118:1519-31; Vithana et al., Investigative Ophthalmology & Visual Science. 2003; 44:4204-9; McGee et al., American Journal Human Genetics. 1997; 61:1059-66). Two mechanisms have been reported to contribute to regulation of expression of the wild-type PRPF31 allele. First, CNOT3 regulates PRPF31 expression via transcriptional repression; in asymptomatic carriers of PRPF31 mutations, CNOT3 is expressed at low levels, allowing higher amounts of wild-type PRPF31 transcripts to be produced and preventing manifestation of retinal degeneration (Venturini et al., PLoS genetics. 2012; 8: e1003040. PMCID: 3493449;Rose et al., Annals Human Genetics. 2013). Second, MSR1 has been identified as a cis regulatory element upstream of the PRPF31. Thus, human genetic variation has provided evidence that augmentation of PRPF31 gene expression can reduce or eliminate vision loss in this disorder.

As described herein, the present inventors have identified RPE cells as the primary cells affected in RNA splicing factor RP; this creates an opportunity to move forward with development of gene augmentation therapy for disease caused by mutations in PRPF31 (see Example 1). To achieve this goal, described herein are AAV vectors for expressing human PRPF31, which can be used to ameliorate the phenotype in human subjects.

The sequence of human PRPF31, also known as U4/U6 small nuclear ribonucleoprotein Prp31, is available in GenBank at Accession Nos. NM_015629.3 (nucleic acid) and NP_056444.3 (Protein). Subjects having RP associated with mutations in PRPF31 can be identified by methods known in the art, e.g., by sequencing the PRPF31 gene (NG_009759.1, Range: 5001 to 21361) or NC_000019.10 Reference GRCh38.p2 Primary Assembly, Range 54115376 to 54131719). A large number of mutations in affected individuals have been identified; see, e.g., Villanueva et al. Invest Ophthalmol Vis Sci, 2014; Dong et al. Mol Vis, 2013; Lu F, et al. PLoS One, 2013; Utz et al. Ophthalmic Genet, 2013; and Xu F, et al. Mol Vis, 2012; Saini et al., Exp Eye Res. 2012 November; 104:82-8; Rose et al., Invest Ophthalmol Vis Sci. 2011 Aug. 22; 52(9):6597-603; Audo et al., BMC Med Genet. 2010 Oct. 12; 11:145; and Tanackovic and Rivolta, Ophthalmic Genet. 2009 June; 30(2):76-83.

Thus described herein are targeted expression vectors for in vivo transfection and expression of a polynucleotide that encodes a PRPF31 polypeptide as described herein, in RPE cells, e.g., primarily or only in RPE cells. Expression constructs of such components can be administered in any effective carrier, e.g., any formulation or composition capable of effectively delivering the component gene to cells in vivo. Approaches include insertion of the gene in viral vectors, including recombinant retroviruses, adenovirus, adeno-associated virus, lentivirus, and herpes simplex virus-1, alphavirus, vaccinia virus, or recombinant bacterial or eukaryotic plasmids; preferred viral vectors are adeno-associated virus type 2 (AAV2). Viral vectors transfect cells directly; plasmid DNA can be delivered naked or with the help of, for example, cationic liposomes (lipofectamine) or derivatized (e.g., antibody conjugated), cationic dendrimers, inorganic vectors (e.g., iron oxide magnetofection), lipidoids, cell-penetrating peptides, cyclodextrin polymer (CDP), polylysine conjugates, gramacidin S, artificial viral envelopes or other such intracellular carriers, as well as direct injection of the gene construct or CaPO4 precipitation carried out in vivo.

An exemplary approach for in vivo introduction of nucleic acid into a cell is by use of a viral vector containing nucleic acid, e.g., a cDNA. Infection of cells with a viral vector has the advantage that a large proportion of the targeted cells can receive the nucleic acid. Additionally, molecules encoded within the viral vector, e.g., by a cDNA contained in the viral vector, are expressed efficiently in cells that have taken up viral vector nucleic acid.

Viral vectors can be used as a recombinant gene delivery system for the transfer of exogenous genes in vivo, particularly into humans. These vectors provide efficient delivery of genes into cells, and in some cases the transferred nucleic acids are stably integrated into the chromosomal DNA of the host. Protocols for producing recombinant viruses and for infecting cells in vitro or in vivo with such viruses can be found in Ausubel, et al., eds., Gene Therapy Protocols Volume 1: Production and In Vivo Applications of Gene Transfer Vectors, Humana Press, (2008), pp. 1-32 and other standard laboratory manuals.

A preferred viral vector system useful for delivery of nucleic acids is the adeno-associated virus (AAV). Adeno-associated virus is a naturally occurring defective virus that requires another virus, such as an adenovirus or a herpes virus, as a helper virus for efficient replication and a productive life cycle. (For a review see Muzyczka et al., Curr. Topics in Micro and Immunol. 158:97-129 (1992)). AAV vectors efficiently transduce various cell types and can produce long-term expression of transgenes in vivo. Although AAV vector genomes can persist within cells as episomes, vector integration has been observed (see for example Deyle and Russell, Curr Opin Mol Ther. 2009 August; 11(4): 442-447; Asokan et al., Mol Ther. 2012 April; 20(4): 699-708; Flotte et al., Am. J. Respir. Cell. Mol. Biol. 7:349-356 (1992); Samulski et al., J. Virol. 63:3822-3828 (1989); and McLaughlin et al., J. Virol. 62:1963-1973 (1989)). AAV vectors, particularly AAV2, have been extensively used for gene augmentation or replacement and have shown therapeutic efficacy in a range of animal models as well as in the clinic; see, e.g., Mingozzi and High, Nature Reviews Genetics 12, 341-355 (2011); Deyle and Russell, Curr Opin Mol Ther. 2009 August; 11(4): 442-447; Asokan et al., Mol Ther. 2012 April; 20(4): 699-708. AAV vectors containing as little as 300 base pairs of AAV can be packaged and can produce recombinant protein expression. Space for exogenous DNA is limited to about 4.5 kb. For example, an AAV1, 2, 4, 5, or 8 vector can be used to introduce DNA into RPE cells (such as those described in Maguire et al. (2008). Safety and efficacy of gene transfer for Leber's congenital amaurosis. N Engl J Med 358: 2240-2248. Maguire et al. (2009). Age-dependent effects of RPE65 gene therapy for Leber's congenital amaurosis: a phase 1 dose-escalation trial. Lancet 374: 1597-1605; Bainbridge et al. (2008). Effect of gene therapy on visual function in Leber's congenital amaurosis. N Engl J Med 358: 2231-2239; Hauswirth et al. (2008). Treatment of leber congenital amaurosis due to RPE65 mutations by ocular subretinal injection of adeno-associated virus gene vector: short-term results of a phase I trial. Hum Gene Ther 19: 979-990; Cideciyan et al. (2008). Human gene therapy for RPE65 isomerase deficiency activates the retinoid cycle of vision but with slow rod kinetics. Proc Natl Acad Sci USA 105: 15112-15117. Cideciyan et al. (2009). Vision 1 year after gene therapy for Leber's congenital amaurosis. N Engl J Med 361: 725-727; Simonelli et al. (2010). Gene therapy for Leber's congenital amaurosis is safe and effective through 1.5 years after vector administration. Mol Ther 18: 643-650;Acland, et al. (2005). Long-term restoration of rod and cone vision by single dose rAAV-mediated gene transfer to the retina in a canine model of childhood blindness. Mol Ther 12: 1072-1082; Le Meur et al. (2007). Restoration of vision in RPE65-deficient Briard dogs using an AAV serotype 4 vector that specifically targets the retinal pigmented epithelium. Gene Ther 14: 292-303; Stieger et al. (2008). Subretinal delivery of recombinant AAV serotype 8 vector in dogs results in gene transfer to neurons in the brain. Mol Ther 16: 916-923; and Vandenberghe et al. (2011). Dosage thresholds for AAV2 and AAV8 photoreceptor gene therapy in monkey. Sci Transl Med 3: 88ra54). In some embodiments, the AAV vector can include (or include a sequence encoding) an AAV capsid polypeptide described in WO 2015054653; for example, a virus particle comprising an AAV capsid polypeptide having an amino acid sequence selected from the group consisting of SEQ ID NOs: 1, 3, 5, 7, 9, 11, 13, 15, and 17 of WO 2015054653, and a PRPF31-encoding sequence as described herein. In some embodiments, the AAV capsid polypeptide is as shown in Table 1 of WO 2015054653, reproduced here:

| | Polypeptide | Nucleic Acid |
|---|---|---|
| Node | (SEQ ID NO) | (SEQ ID NO) |
| Anc80 | 1 | 2 |
| Anc81 | 3 | 4 |
| Anc82 | 5 | 6 |
| Anc83 | 7 | 8 |
| Anc84 | 9 | 10 |
| Anc94 | 11 | 12 |
| Anc113 | 13 | 14 |
| Anc126 | 15 | 16 |
| Anc127 | 17 | 18 |

In some embodiments, the AAV capsid polypeptide is an Anc80 polypeptide, e.g., an exemplary polypeptide shown in SEQ ID NO: 19 (Anc80L27); SEQ ID NO: 20 (Anc80L59); SEQ ID NO: 21 (Anc80L60); SEQ ID NO: 22 (Anc80L62); SEQ ID NO: 23 (Anc80L65); SEQ ID NO: 24 (Anc80L33); SEQ ID NO: 25 (Anc80L36); and SEQ ID NO:26 (Anc80L44).

A variety of nucleic acids have been introduced into different cell types using AAV vectors (see for example the references cited above and those cited in Asokan et al., Molecular Therapy (2012); 20 4, 699-708; and Hermonat et al., Proc. Natl. Acad. Sci. USA 81:6466-6470 (1984); Tratschin et al., Mol. Cell. Biol. 4:2072-2081 (1985); Wondisford et al., Mol. Endocrinol. 2:32-39 (1988); Tratschin et al., J. Virol. 51:611-619 (1984); and Flotte et al., J. Biol. Chem. 268:3781-3790 (1993).

Retroviruses can also be used. The development of specialized cell lines (termed “packaging cells”) which produce only replication-defective retroviruses has increased the utility of retroviruses for gene therapy, and defective retroviruses are characterized for use in gene transfer for gene therapy purposes (for a review see Katz et al., Human Gene Therapy 24:914 (2013)). A replication defective retrovirus can be packaged into virions, which can be used to infect a target cell through the use of a helper virus by standard techniques. Examples of suitable retroviruses include pLJ, pZIP, pWE and pEM which are known to those skilled in the art. Examples of suitable packaging virus lines for preparing both ecotropic and amphotropic retroviral systems include ΨCrip, ΨCre, Ψ2 and ΨAm. Retroviruses have been used to introduce a variety of genes into many different cell types, including epithelial cells, in vitro and/or in vivo (see for example Eglitis, et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad. Sci. USA 87:6141-6145; Huber et al. (1991) Proc. Natl. Acad. Sci. USA 88:8039-8043; Ferry et al. (1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol. 150:4104-4115; U.S. Pat. Nos. 4,868,116; 4,980,286; PCT Application WO 89/07136; PCT Application WO 89/02468; PCT Application WO 89/05345; and PCT Application WO 92/07573).

Another viral gene delivery system useful in the present methods utilizes adenovirus-derived vectors. The genome of an adenovirus can be manipulated, such that it encodes and expresses a gene product of interest but is inactivated in terms of its ability to replicate in a normal lytic viral life cycle. See, for example, Berkner et al., BioTechniques 6:616 (1988); Rosenfeld et al., Science 252:431-434 (1991); and Rosenfeld et al., Cell 68:143-155 (1992). Suitable adenoviral vectors derived from the adenovirus strain Ad type 5 dl324 or other strains of adenovirus (e.g., Ad2, Ad3, or Ad7 etc.) are known to those skilled in the art. Recombinant adenoviruses can be advantageous in certain circumstances, in that they are not capable of infecting non-dividing cells and can be used to infect a wide variety of cell types, including epithelial cells (Rosenfeld et al., (1992) supra). Furthermore, the virus particle is relatively stable and amenable to purification and concentration, and as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situ, where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al., supra; Haj-Ahmand and Graham, J. Virol. 57:267 (1986).

In some embodiments, a gene encoding PRPF31 is entrapped in liposomes bearing positive charges on their surface (e.g., lipofectins), which can be tagged with antibodies against cell surface antigens of the target tissue (Mizuno et al., No Shinkei Geka 20:547-551 (1992); PCT publication WO91/06309; Japanese patent application 1047381; and European patent publication EP-A-43075).

In clinical settings, the gene delivery systems for the therapeutic gene can be introduced into a subject by any of a number of methods, each of which is familiar in the art. Although other methods can be used, in some embodiments, the route of choice for delivery of gene therapy vectors to the retina is via sub-retinal injection. This provides access to the RPE and photoreceptor cells of the retina. Different serotypes of AAV have been shown to transduce these cell populations effectively after sub-retinal injection in animal studies (Vandenberghe et al., PLoS One. 2013; 8:e53463. PMCID: 3559681; Vandenberghe and Auricchio, Gene Therapy. 2012; 19:162-8; Vandenberghe et al., Science translational medicine. 2011; 3:88ra54; Dinculescu et al., HumGene Ther. 2005; 16:649-63; Boye et al., Mol Ther. 2013; 21:509-19; Alexander and Hauswirth, Adv Exp Med Biol. 2008; 613:121-8). The sub-retinal injection approach is being used in the ongoing clinical trials of gene augmentation therapy for retinal degeneration caused by mutations in the RPE65 and CHM genes genetic disease (Maguire et al., New England Journal of Medicine. 2008; 358:2240-8; Bainbridge et al., New England Journal of Medicine. 2008; 358:2231-9; Cideciyan et al., Proceedings National Academy Sciences USA. 2008; 105:15112-7; Maguire et al., Lancet. 2009; 374:1597-605; Jacobson et al., Archives Ophthalmology. 2012; 130:9-24; Bennett et al., Science translational medicine. 2012; 4:120ra15; MacLaren et al., Lancet. 2014; 383:1129-37). Sub-retinal injections can be performed using a standard surgical approach (e.g., as described in Maguire et al., 2008 supra; Bainbridge et al., 2008 supra; Cideciyan et al., 2008 supra; MacLaren et al., 2014 supra).

The pharmaceutical preparation of the gene therapy construct can consist essentially of the gene delivery system (viral vector and any associated agents such as helper viruses, proteins, lipids, and so on) in an acceptable diluent, or can comprise a slow release matrix in which the gene delivery vehicle is embedded. Alternatively, where the complete gene delivery system can be produced intact from recombinant cells, e.g., retroviral vectors, the pharmaceutical preparation can comprise one or more cells, which produce the gene delivery system.

### EXAMPLES

The invention is further described in the following examples, which do not limit the scope of the invention described in the claims.

### Example 1. Mutations in Pre-mRNA Processing Factors 3, 8 and 31 Cause Dysfunction of the Retinal Pigment Epithelium

### Introduction

The spliceosome is a ubiquitous, dynamic ribonucleoprotein macromolecule required for removing introns from a nascent RNA1. Mutations that cause autosomal dominant retinitis pigmentosa (RP) have been identified in 6 genes that encode proteins (PRPF3, PRPF4, PRPF6, PRPF8, PRPF31, and SNRNP200), which are found in the U4/U6.U5 tri-snRNP2. In aggregate, mutations in these genes are the second most common cause of dominant RP3-5. Defined by progressive, late-onset vision loss, RP is the most common form of inherited retinal degeneration, affecting approximately 1:3500 individuals worldwide6. It is genetically heterogeneous, and displays all three modes of Mendelian inheritance7. Affected tissues include the neural retina, retinal pigment epithelium (RPE), and choroid4. Since the components of the spliceosome are ubiquitously expressed in every cell type, it is not clear why mutations in these splicing factors cause only non-syndromic RP. Further, the specific cell type(s) in the retina affected by these mutations has not yet been identified.

We have previously reported the characterization of mouse models of RNA splicing factor RP due to mutations in the PRPF3, PRPF8 and PRPF31 genes, including Prpf3, Prpf8 and Prpf31 knockout mice, and Prpf3-T494M and Prpf8-H2309P knockin mice8, 9. Based on results from studies of these mouse models, and data from human studies, it is believed that mutations in PRPF3 and PRPF8 cause dominant disease via gain-of-function or dominant-negative mechanisms, while mutations in PRPF31 cause disease via haploinsufficiency9-11. Morphological changes in the aging RPE, but not the neural retina, of the Prpf3-T494M and Prpf8-H2309P knockin mice and Prpf31^{+/-} mice were of particular interest, where we observed the loss of basal infoldings, the formation of basal deposits beneath the RPE and vacuolization in the cytoplasm. These RPE degenerative changes were observed in heterozygous Prpf3^{T494M/+}, Prpf8^{H2309P/+} and Prpf31^{+/-} mice, and were more pronounced in homozygous Prpf3^{T494M/T494M} and Prpf8^{H2309P/H2309P} knockin mice8.

The RPE is vital for the overall well-being of the retina12. The daily elimination of spent photoreceptor outer segment extremities (POS) is a highly coordinated process, and phagocytosis of shed POS occur on a rhythmic basis¹³. Some receptors implicated in POS phagocytosis also participate in overall retinal adhesion and its physiological rhythm¹⁴. Peak phagocytosis and retinal adhesion occur approximately 2 and 3.5 hours after light onset, respectively, and are at their minimum levels roughly 10 hours later13, 15, 14. The RPE is a professional macrophage where binding and internalization of a substrate is coordinated by receptors on the RPE cell and ligands in the interphotoreceptor matrix bridging the RPE cell and phosphatidylserines at the POS surface, respectively¹⁶. Some receptors are common between phagocytosis and adhesion, but they use different ligands^{13, 14, 15, 17}. A loss of regulation of any of these important components of phagocytosis leads to vision loss in human disease as well as in rodent models13, 18-20.

Here we report results of studies of RPE phagocytosis and adhesion for the Prpf3^{T494M/T494M}, Prpf8^{H2309P/H2309P} and Prpf31^{+/-} mouse models. Specifically, we measured phagocytosis in primary RPE cultures from 2-week-old mice. Results show a deficiency in phagocytosis, which we also demonstrate in the human RPE cell line, ARPE-19, following shRNA-mediated knockdown of PRPF31. Additionally, a loss of diurnal rhythmicity of phagocytosis and adhesion were detected in vivo. Interestingly, localization of key factors known to be involved in phagocytosis by RPE cells is modified. We conclude that the RPE is likely to be the primary site of pathogenesis in RNA splicing factor RP.

### Materials and Methods

### Animals

Animal research was performed under the protocols approved by the Institutional Animal Care and Use Committees at the Massachusetts Eye and Ear Infirmary and the Charles Darwin Animal Experimentation Ethics Committee from the Université Pierre et Marie Curie-Paris. An equal number of male and female mice were used in each of the following experiments.

### Primary RPE Cell Culture

RPE cells from 9-10-day-old animals were isolated as described13. Briefly, eyecups were digested with 2 mg/ml of hyaluronidase (Sigma) and the neural retina was gently peeled from the eyecup. RPE were peeled from the Bruch's membrane following digestion with 1 mg/ml trypsin (Invitrogen) and seeded onto 5-mm glass coverslips. Cells were grown to confluency for 5-10 days in DMEM with 10% FBS at 37° C., 5% CO₂.

### Primary Peritoneal Macrophage Cell Culture

Resident peritoneal macrophages were isolated as previously described²¹. Euthanized mice were pinned down to a dissection board and the fur dampened using 70% ethanol in a horizontal flow hood. The skin was delicately separated from the peritoneal wall using forceps and scissors. 5 mL of sterile PBS were injected in the abdominal cavity and the belly massaged or the whole body shaken gently for 20-30 seconds. PBS was collected slowly from the cavity and samples from 2 to 3 different animals pooled. Cells were spun for 10 min at 300 g and resuspended in 1 mL RPMI with 10% FBS. Cells were seeded in 96-well plates at 100,000-200,000 cells per well and allowed to adhere for 2 hours. Plates were shaken and wells rinsed once using sterile PBS. Cells were maintained in medium for 2-3 days at 37° C., 5% CO₂.

### Generation of Stable shRNA-PRPF31 Knockdown ARPE-19 and J774.1 Cell Lines and Cell Viability Assay

Three shRNAs were designed to human PRPF31 or mouse Prpf31 and cloned into pCAG-mir30 vector containing a puromycin resistance gene. The sequences for these shRNAs are as follows: human shRNA1-5′-TGCTGTTGACAGTGAGCGAGCAGATGAGCTCTTAGCTGATTAGTGAAGCC ACAGATGTAATCAGCTAAGAGCTCATCTGCCTGCCTACTGCCTCGGA-3′ (SEQ ID NO:27), human shRNA2-5′-TGCTGTTGACAGTGAGCGAACCCAACCTGTCCATCATTATTAGTGAAGCC ACAGATGTAATAATGATGGACAGGTTGGGTGTGCCTACTGCCTCGGA-3′ (SEQ ID NO:28), and human shRNA3-5′-TGCTGTTGACAGTGAGCGAGCTGAGTTCCTCAAGGTCAAGTAGTGAAGCC ACAGATGTACTTGACCTTGAGGAACTCAGCCTGCCTACTGCCTCGGA-3′ (SEQ ID NO:29); mouse shRNA1-5′-TGCTGTTGACAGTGAGCGCTCAGTCAAGAGCATTGCCAAGTAGTGAAGCC ACAGATGTACTTGGCAATGCTCTTGACTGAATGCCTACTGCCTCGGA-3′ (SEQ ID NO:30), mouse shRNA2-5′-TGCTGTTGACAGTGAGCGACCTGTCTGGCTTCTCTTCTACTAGTGAAGCCA CAGATGTAGTAGAAGAGAAGCCAGACAGGGTGCCTACTGCCTCGGA-3′ (SEQ ID NO:31), and mouse shRNA3-5′-TGCTGTTGACAGTGAGCGAGCCGAGTTCCTCAAGGTCAAGTAGTGAAGCC ACAGATGTACTTGACCTTGAGGAACTCGGCCTGCCTACTGCCTCGGA-3′ (SEQ ID NO:32). We also cloned an shRNA to green fluorescence protein into this vector as a non-targeted control (5′-TGCTGTTGACAGTGAGCGCTCTCCGAACGTGTATCACGTTTAGTGAAGCCA CAGATGTAAACGTGATACACGTTCGGAGATTGCCTACTGCCTCGGA-3′ (SEQ ID NO:33)). The shRNA-containing vectors were linearized with PstI and transfected into separate ARPE-19 (human RPE cell line, ATCC) or J774A.1 (mouse macrophage cell line, ATCC) cultures using the Amaxa electroporation kit V (Amaxa). Transfected cells were transferred to 6-well plates and 2 ml of culture medium (1:1 DMEM: F-12 with 10% FBS). Transfected cells were grown overnight at 37° C., 5% CO₂. Stable cell lines were selected with the addition of 1 (ARPE-19) to 1.25 (J774A.1) μg/ml of puromycin (Sigma) 24 hours following transfection. Media and puromycin were refreshed every 2 days for 10 days. Following selection, the four ARPE-19 and four J774A.1 knockdown lines were grown to confluence. To determine knockdown efficiency, stable lines were transiently transfected with either V5-tagged PRPF31 in ARPE-19 cells or V5-tagged Prpf31 cloned in a Gateway Destination vector (Invitrogen). Western blot was performed and V5-tagged PRPF31 was quantified using an Odyssey Infrared Imager (Li-Cor). Cell viability assays were performed using the Cell Titer-Glo Luminescent Cell Viability Assay (Promega) according to manufacturer's recommendations. Briefly, ARPE-19 cells were seeded at a density of 1,000 cells/well of a 96-well cell culture plate (Corning, Cat #3904). Cells were grown for 3 days in DMEM with 10% FBS at 37° C., 5% CO₂. Following this period, cell viability was measured by luminescence, and statistical significance was determined using the Student's t-test.

### In Vitro Phagocytosis Assays

Photoreceptor outer segments were isolated from porcine eyes obtained fresh from the slaughterhouse and covalently labeled with FITC dye (Invitrogen) for in vitro phagocytosis assays as previously described13. Confluent cultured RPE cells were challenged with ˜10 FITC-POS per cell for 1.5 hours. Non-specifically bound POS were thoroughly removed with three washes in PBS with 1 mM MgCl₂ and 0.2 mM CaCl₂. To measure internalized POS, some wells were incubated with trypan blue for 10 min to quench fluorescence of surface-bound FITC-labeled POS as previously described²⁶. Cells were fixed with ice-cold methanol and nuclei were counterstained with Hoechst 33258 (Invitrogen) or DAPI (Euromedex). Cells were imaged using a Nikon Ti2 or a Leica DM6000 Fluorescent microscope at 20×. For RPE primary cultures, FITC/DAPI ratios were calculated on all picture fields, corresponding to the number of POS per cell. FITC-POS were counted on a per cell basis for 100 cells and the average determined for three wells for ARPE-19. For peritoneal macrophages, FITC-POS and DAPI-labeled nuclei were quantified by fluorescence plate reading (Infinite M1000, Magellan 6 software, Tecan). Binding ratios were calculated by subtracting results obtained in internalization wells (trypan blue-treated) from total phagocytosis (untreated) wells. This was performed for three to six independent assays and significance was determined using the Student's t-test (P<0.05).

Prior to phagocytosis, confluent cultures of the stable knockdown J774A.1 lines were opsonized using Zymosan A Bioparticles Opsonizing Reagent (Life Technologies) according to the manufacturer's protocol. Following opsonization, 1 μg of Zymosan A Bioparticles reconstituted in culture medium were applied to each culture well of a 96-well plate. The cultures were incubated at 37° C., 5% CO₂ for 1 hour. Fixation and determination of phagocytosis levels were performed as described above.

### In Vivo Diurnal Rhythm Assays

Mice were euthanized at 2 hours before light onset (−2), at light onset (0), and 2, 4, and 8 hours (+2, +4, +8) after light onset, and processed for either electron microscopy or paraffin embedding as previously described13, 15. For electron microscopy all reagents were purchased from Electron Microscopy Sciences. Mice were perfused with 2% glutaraldehyde+2% paraformaldehyde, and eyecups were transferred to perfusion buffer with the addition of 0.2 M sodium cacodylate buffer. Sixty to eighty nanometer ultrathin sections were stained with lead citrate/uranyl acetate and early phagosomes were counted from 200 nM out from the optic nerve. An early phagosome is counted if it meets the following criteria: 1) it is contained within the cytoplasm of the RPE and 2) has visible lamellar structure. For light microscopy, eyecups were fixed in formaldehyde/ethanol/acetic acid and embedded in paraffin using Ottix Plus solvent substitute (DiaPath). Five-micrometer sections were cut and the paraffin was removed using SafeSolv solvent substitute. The sections were rehydrated and incubated in 5% H₂O₂ in 1× SSC for 10 minutes under illumination to bleach pigments. After blocking non-specific signals using 10% BSA in 1× TBS, sections were stained with an anti-rhodopsin antibody (Millipore) and anti-mouse IgG-AlexaFluor 488 (Invitrogen). Nuclei were stained with DAPI, and slides mounted with Mowiol (prepared according to standard procedures). Image stacks were acquired on an Olympus FV1000 inverted confocal microscope with a 60× oil objective, a 4-time zoom and 0.41-μm step size scans and processed using the Adobe Photoshop CS6 software. Areas of at least 100 μm of uninterrupted retina/RPE were counted on 10-scan stacks. In each experiment series, phagosome counts were normalized to length of retina and averaged. Significance was determined using the Student's t-test (P<0.05) and N=2-5 for all experiments.

### In Vivo Retinal Adhesion Assays

We performed in vivo retinal adhesion assays as described¹⁴. Briefly, lens and cornea were removed from eyecups immediately postmortem in Hanks saline buffer with calcium and magnesium. A radial cut was made to the optic nerve, and the neural retina was gently peeled from the flattened eyecup. Neural retina samples were lysed in 50 mM Tris-HCl (pH 7.5), 2 mM EDTA, 150 mM NaCl, 1% Triton X-100, 0.1% SDS, and 1% Nonidet P-40, with addition of a protease inhibitors cocktail (Sigma) and 1 mM PMSF. Proteins from cleared supernatants were quantified using the Bradford assay and equal concentrations were immunoblotted for RPE65 (Abcam or Millipore) and beta-actin (Abcam or Sigma). Melanin pigments were extracted from the insoluble neural retina pellet with 20% DMSO, 2N NaOH. Samples and commercial melanin standards (Sigma) were quantified by measuring absorbance at 490 nM. Pigment abundance was normalized to protein concentration in each sample to account for different tissue yields. Bands from immunoblots were quantified using the ImageJ software v1.46r using a common sample on all blots as reference, signals were then averaged. Significance was determined using the Student's t-test (P<0.05) and N=3-6 for all experiments.

### Immunofluorescence Microscopy

For cryosections, eyecups were fixed in 2% paraformaldehyde and incubated in 30% sucrose overnight at 4° C. Eyecups were embedded in O.C.T. Compound (Sakura) and 10-μm sections were cut. Sections were individually incubated with primary antibodies against av integrin (BD Biosciences), β5 integrin (Santa Cruz Biotechnology), MerTK (FabGennix), Mfg-E8 and Gas6 (R&D Systems), FAK clone 2A7 (Millipore), and Protein S (Sigma), followed by IgG-AlexaFluor 488 (Invitrogen). Nuclei were stained with DAPI, and mounted with Fluoromount (Electron Microscopy Sciences). Images were taken with a Nikon Eclipse Ti inverted fluorescence microscope using an oil immersion 60× objective. Images were processed with NIS-Elements AR software (Nikon).

For paraffin sections, animals were sacrificed at the time of the phagocytic peak and eyes were fixed in Davidson fixative for three hours at 4° C., then lens and cornea were removed. Eyecups were embedded in paraffin and 5-μm sections were cut. Sections were treated as described in the In Vivo Diurnal Rhythm Assays section and individually incubated with primary antibodies against av integrin (Covance), β5 integrin (Santa Cruz Biotechnology), Mfg-E8, MerTK and Gas6 (R&D Systems), FAK clone 2A7 (Millipore), and Protein S (Novus Biologicals), followed by secondary antibody incubation with IgG-AlexaFluor 488 (Invitrogen). Nuclei were stained with DAPI, and slides mounted with Mowiol. Images were taken with a Leica DM6000 B Epifluorescence microscope using a 40× oil immersion objective. Images were processed with ImageJ v1.46r and Photoshop CS6 software.

### Results

### RPE phagocytosis is Decreased in Prpf-Mutant Mice

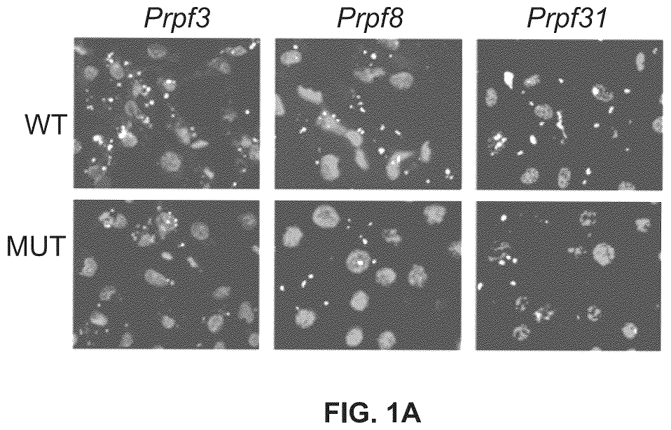

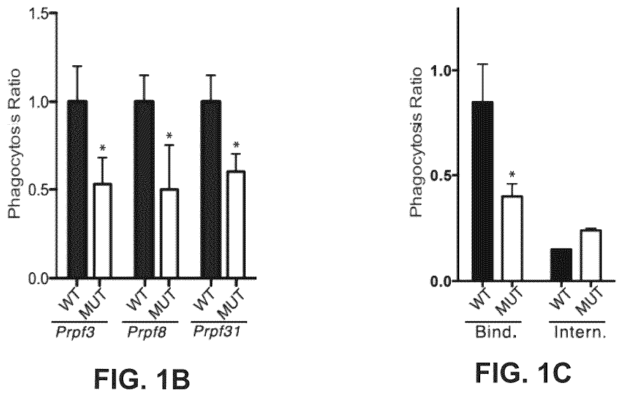

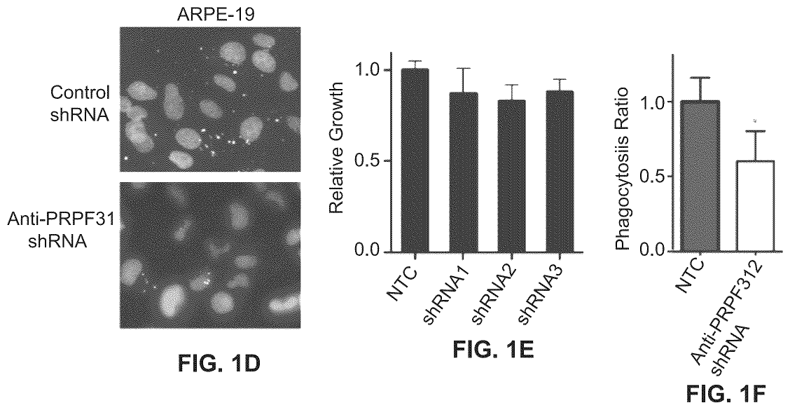

In our original characterization of the Prpf-mutant mice, electron microscopy identified morphological changes in the RPE of 1 to 2-year-old mutants8. Here, we set out to determine if functional changes precede the observed morphological changes. Since the RPE maintains phagocytic activity in culture, we established independent primary RPE cultures from 9-10-day-old Prpf3^{T494M/T494M}, Prpf8^{H2309P/H2309P}, Prpf31^{+/-} mice, and their corresponding littermate controls. Once the cultures were confluent, we used FITC-labeled porcine POS and measured the phagocytosis following a 1.5-hour incubation. FIG. 1A (panels 1-3) shows representative images of primary cultures illustrating the POS binding/uptake of RPE cells from the Prpf-mutant mice and their littermate controls, and demonstrating the qualitative deficiency in phagocytosis by the mutant mice. In all three mutant models, a 37-48% decrease in phagocytosis was observed (N=3-5, P<0.05) (FIG. 1B). To account for non-specific binding of POS to the coverslips, we ran a negative control, in which the phagocytosis assay was performed on coverslips that did not contain cells. We did not observe any non-specific adhesion of the POS to the coverslips (data not shown).

We investigated if a specific step of phagocytosis between binding and internalization is preferentially perturbed in Prpf31^{+/-} RPE primary cultures. After performing a 1.5-hour phagocytic challenge, we treated the cells to quench the surface (bound POS) fluorescence in order to quantify solely internalized POS. POS binding was significantly reduced by 53±11% in mutant cells (N=2-5, P<0.05), whereas there was no significant difference in POS internalization rates between wild-type and mutant RPE cultures (FIG. 1C).

Currently, there are 64 known pathogenic mutations in PRPF31, of which many result in a frameshift and are degraded via the non-sense mediated decay pathway2, 10, 11, 22. ARPE-19 is a spontaneously immortalized human RPE cell line that is amenable to transfection and retains the ability to phagocytose23. To test whether mutations in the splicing factors also affect phagocytosis in a human RPE model, we created three stable ARPE-19 cell lines with shRNA-mediated knockdown of PRPF31 using 3 distinct shRNAs directed against the 5′, 3′ and middle regions of the transcript (FIG. 1D). We also generated a fourth stable cell line with an shRNA directed against the green fluorescent protein to use as a control. In each of the three PRPF31 shRNA stable cell lines we achieved approximately 60-95% knockdown of PRPF31 (data not shown). Cell viability assays of the shRNA-knockdown and non-targeted control ARPE-19 cells showed that no significant decrease occurred in association with the knockdown of PRPF31 (FIG. 1E). Phagocytosis was decreased by approximately 40% in each line tested, compared to the non-targeted control shRNA line (FIG. 1F). As with the phagocytosis assay performed on primary RPE, we also performed a negative control assay, and did not observe any non-specific adhesion of the POS to the coverslips (data not shown).

In order to determine if disruption of the phagocytic machinery is an RPE-specific mechanism, or can be observed in other phagocytic cells, we knocked down Prpf31 in the mouse macrophage cell line, J774A.1. Similar to the knockdown studies in the ARPE-19 cell line, three distinct shRNAs were directed to the 5′-, 3′-termini and middle of the transcript. We used the same control shRNA as the previous studies. In each of the stable Prpf31 cell lines, we achieved approximately 45-70% knockdown of Prpf31 (Supplemental FIG. 1A). We did not observe any phagocytosis deficiency in any of the lines tested (Supplemental FIG. 1B). To ensure we did not observe non-specific POS adhesion, we performed a negative control assay as for the previous experiment series (data not shown). Identical experiments were repeated on mouse primary peritoneal macrophages isolated from Prpf31^{+/-} mice. Interestingly, neither step of phagocytosis, i.e. binding or internalization, nor total phagocytosis was affected in Prpf31-mutant compared to wild-type macrophages (Supplemental FIG. 1C).

### The Diurnal Rhythmicity of Phagocytosis is Disrupted

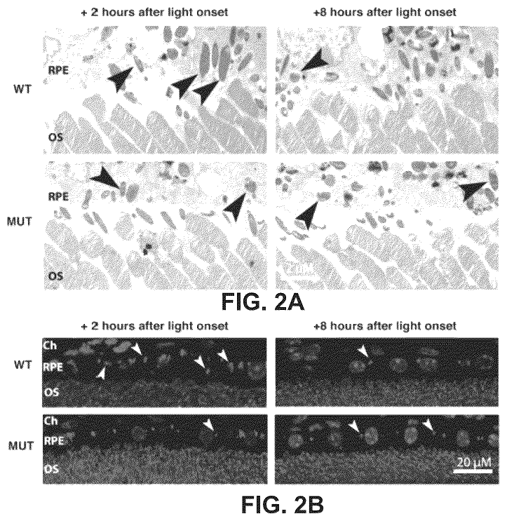

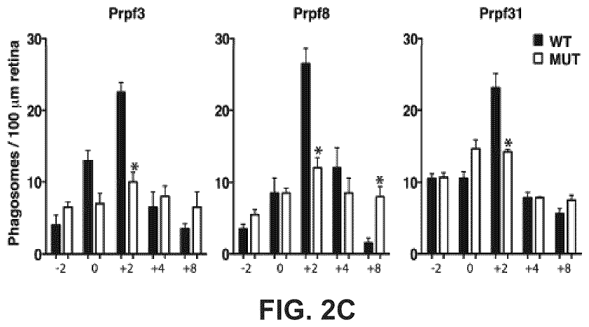

Phagocytosis of shed POS by the RPE follows a strong diurnal, synchronized rhythm peaking at 2 hours after light-onset and remaining relatively inactive for the remainder of the day¹³. We measured phagocytosis in vivo at 5 time-points throughout the light cycle using either electron microscopy (FIG. 2A, Prpf3- and Prpf8-mutants) or immunofluorescence (FIG. 2B, Prpf31-mutant), both recognized techniques to assess the RPE phagocytic rhythm13, 15. For Prpf3 and Prpf8 control and mutant mice we counted early phagosomes containing lamellar structures on electron micrographs (FIG. 2A, arrowheads, insets show lamellar structures). Phagocytosis rhythmicity was determined in Prpf31^{+/-} mice using paraffin embedding and staining for rhodopsin, and we counted phagosomes present in the RPE cell layer (FIG. 2B, arrowheads). We observed a phagocytosis burst at 2 hours after light onset in all control mice, identifying 22-26 phagosomes per 100 μm of retinal section (FIG. 2C, +2 time-point). In contrast, mutant mice only displayed 10-14 phagosomes at the same peak time-point. During the rest of the light:dark cycle, phagocytosis levels remain relatively low in control mice (“off-peak hours”, 2-12 phagosomes/100 μm retina), and these levels are generally increased in mutant mice (6-14 phagosomes/100 μm retina). These results show a decrease in the phagocytic peak intensity in all three types of mutant mice, with a spreading of the time of the peak that lasts longer in Prpf3- and Prpf8-mutants and starts earlier in Prpf31-mutants. Further, the Prpf8-mutants have significantly more phagosomes at the off-peak time point (+8 hrs), relative to the WT controls.

### Decreased Retinal Adhesion is Observed at the Peak Time-Point

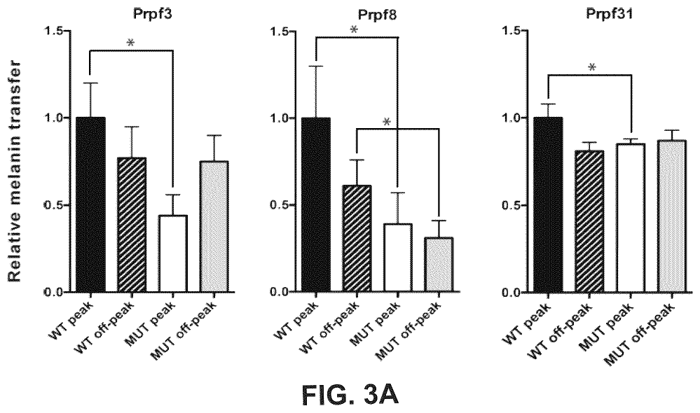

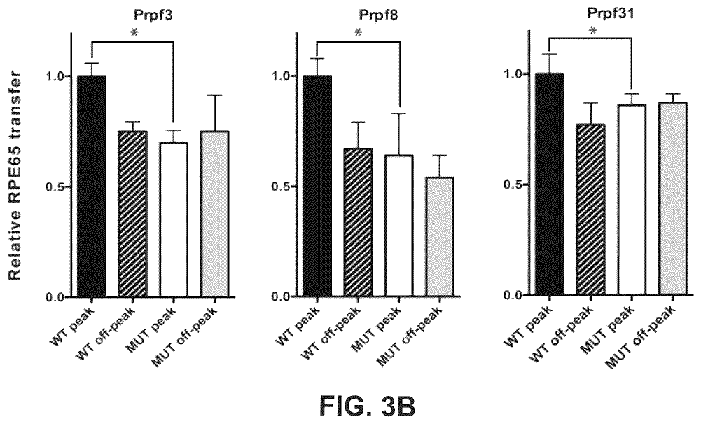

Adhesion between the RPE apical microvilli and distal tips of the POS is known to follow a synchronized rhythm with maximum strength occurring 3.5 hours after light onset, slightly after the phagocytic peak14, 15. Adhesion can be determined by peeling the retina from a flattened eyecup immediately after euthanasia, then quantifying both the RPE melanin content and apical RPE protein markers, such as RPE65, transferred to the retina. Using this method, we assessed adhesion in Prpf-mutant mice and littermate controls at 3.5 and 8.5 hours after light onset (peak and off-peak adhesion, respectively). RPE adhesion was quantified first using a standard melanin quantification procedure¹⁴, then western blotting for the presence of RPE65 to confirm the melanin results. We noted a decrease of 56±16% (N=6, p<0.05, variation is equal to the standard deviation) of the melanin content in the Prpf3^{T494M/T494M} at peak time and no significant change in adhesion at the off-peak time-point (FIG. 3A). Western blot analysis confirmed this observation with a 30±2% decrease in peak adhesion (FIG. 3B). Melanin quantification in Prpf8^{H2309P/H2309P} mice showed that adhesion was significantly decreased by 61±28% at the peak time-point, and 51±16% at the off-peak time point (N=6, P<0.05 for both time-points) (FIG. 3A). Western blot analysis, however, confirmed a significant 36±11% decrease only at the peak time-point (FIG. 3B). In the Prpf31^{+/-} mice, a 15±1% decrease was observed at the peak time-point (FIG. 3A), and confirmed by immunoblot analysis (N=3-7, P<0.05 for both panels) (FIG. 3B, 14±1%).

### Localization of Phagocytosis and Adhesion Markers

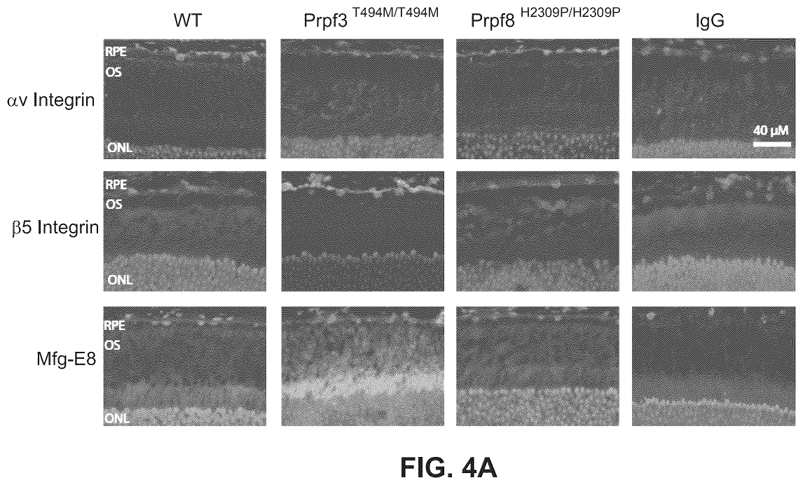

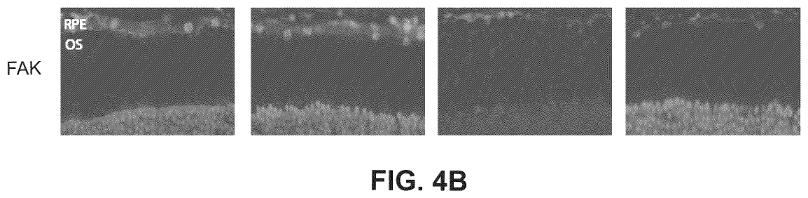

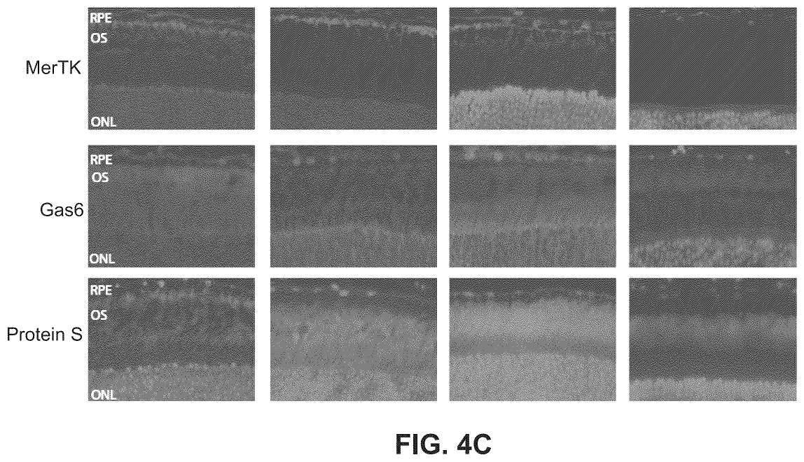

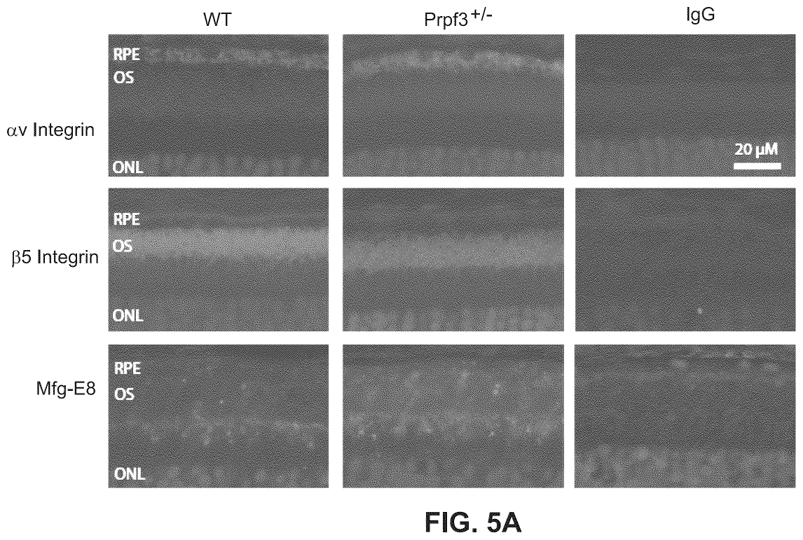

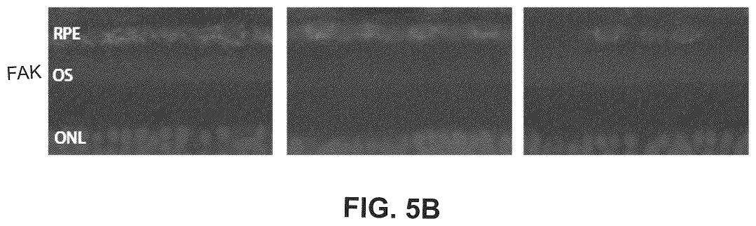

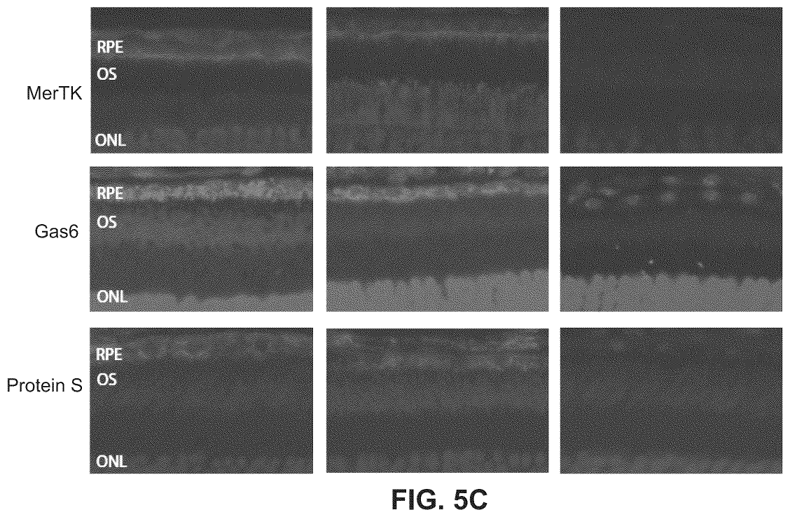

RPE cells are highly polarized, and their function is dependent upon this polarity24. The specific localization of many proteins expressed in the RPE is important, and irregularities in localization may cause retinal dystrophies such as RP or Best disease25, 26. Given the disruption of the diurnal rhythm of both phagocytosis and adhesion in all three Prpf-mutant mouse models, we set out to characterize the localization of the proteins that are known to be important for these processes. Protein localization was assayed on cryosections for Prpf3- and Prpf8-mutant mice (FIG. 4), and on paraffin sections for Prpf31-mutant mice (FIG. 5).

As shown previously, the main phagocytic receptors (αvβ5 integrin and MerTK) localize at the RPE apical surface27, while their ligands can be expressed throughout the POS and RPE28. Interestingly, extracellular ligands expressed in the interphotoreceptor matrix can be synthesized by both RPE and photoreceptor cells.

It has been shown that αvβ5-integrin with its associated ligand Mfg-E8 (milk fat globule-EGF8) are important for phagocytosis and are responsible for the diurnal rhythmicity of this function13, 15. In addition, αvβ5-integrin participates in retinal adhesion and its rhythm, but with a ligand different from Mfg-E8^{14, 15, 17}. αv integrin subunits associate in complexes with several β integrin subunits in RPE cells¹⁴, therefore it is more relevant to analyze the expression of β5 integrin subunits. Thus, we probed for the αv and β5 subunits of the αvβ5 integrin receptor separately. In wild-type tissues each integrin localized primarily to the apical side of the RPE, with some expression throughout the RPE cells. In all 3 Prpf-mutant tissues, no change was observed in αv-integrin localization (FIGS. 4A, 5A). In contrast, β5 integrin localized primarily to the basal side of the RPE in the Prpf3- and Prpf31-mutant tissues, while it displayed expression equally throughout the RPE in Prpf8-mutant RPE cells. We did not observe a change in the localization of Mfg-E8 in either the RPE or POS, but seems to be more expressed in both Prpf8 and 31 mutants.

The downstream signaling protein FAK (focal adhesion kinase) provides a sequential activation link between αvβ5 integrin and MerTK receptors both in vitro and in vivo29, 30, 13. FAK is found throughout the RPE, and no change to this pattern was observed in the Prpf3- or the Prpf31-mutant mice (FIG. 4B, 5B). Prpf8-mutant mice, however, showed FAK localization to the basal side of the RPE.

Phagocytosis is driven by the timely activation of MerTK via phosphorylation at the time of the activity peak13, 31, 32. Gas6 and Protein S are ligands of MerTK that can stimulate uptake of shed outer segments in vitro33. Both ligands are necessary to the internalization of POS as double knockout animals recapitulate the rapid retinal degeneration occurring in rats in whose MerTK receptors are absent34. MerTK expression in wild-type tissues is localized to both the apical and basal membranes of the RPE, whereas MerTK is localized solely to the apical side of Prpf31-mutant RPE cells (FIGS. 4C, 5C). The first MerTK ligand Gas6 localizes to the POS and apical layer of the RPE in wild-type tissues. A decrease of expression is observed in the Prpf3-mutant mice POS, with diffuse expression seen throughout the RPE. Prpf8-mutant mice maintain Gas6 expression in the POS, but appear to lose apical localization in the RPE, also showing a diffuse expression throughout the RPE. No localization changes can be observed in Prpf31-mutant mice. The expression of the second MerTK ligand Protein S is localized specifically to the POS in wild-type and all Prpf-mutant mice (FIGS. 4C, 5C).

### Discussion

Here, we report the first functional characterization of the RPE in mice with mutations in the RNA splicing factors Prpf3, 8, and 31. As we have previously reported, the mutant mice do not experience photoreceptor degeneration, but rather morphological changes in the RPE⁸. Since RNA splicing factor RP is a late onset disease, these results are not surprising and the models afford us the ability to study the mechanisms leading to the onset of disease. Our results demonstrate that the RPE is likely to be the primary cell type affected by mutations in these 3 RNA splicing factors in the mouse, and in humans given the similar phagocytic deficiency observed in PRPF31-knockdown human ARPE-19 cells. While the exact mechanism of disease pathogenesis remains to be identified, these data allow for research to be focused on the RPE. For example, the identification of the RPE as the primary cell type affected in these disorders will make it possible to extend these studies to human cells, as it is now possible to generate RPE cells from human induced pluripotent stem cells (hiPSCs) of patients with inherited retinal diseases⁴²⁻⁴⁵.

### REFERENCES FOR EXAMPLE 1

[1] Will CL, Luhrmann R: Spliceosome Structure and Function. Cold Spring Harbor Perspectives in Biology 2011, 3.
[2] Liu MM, Zack DJ: Alternative splicing and retinal degeneration. Clinical Genetics 2013, 84: 142-149.
[3] Daiger SP, Bowne SJ, Sullivan LS: Perspective on genes and mutations causing retinitis pigmentosa. Archives of Ophthalmology 2007, 125:151-158.
[4] Hartong DT, Berson EL, Dryja TP: Retinitis pigmentosa. The Lancet 2006, 368:1795-809.
[5] Sullivan LS, Bowne SJ, Reeves MJ, Blain D, Goetz K, NDifor V, Vitez S, Wang X, Tumminia SJ, Daiger SP: Prevalence of Mutations in eyeGENE Probands With a Diagnosis of Autosomal Dominant Retinitis Pigmentosa. Investigative Ophthalmology & Visual Science 2013, 54:6255-61.
[6] Nishiguchi KM, Rivolta C: Genes Associated with Retinitis Pigmentosa and Allied Diseases Are Frequently Mutated in the General Population. PLoS ONE 2012, 7.
[7] Neveling K, Collin RWJ, Gilissen C, van Huet RAC, Visser L, Kwint MP, Gijsen SJ, Zonneveld MN, Wieskamp N, de Ligt J, Siemiatkowska AM, Hoefsloot LH, Buckley MF, Kellner U, Branham KE, den Hollander AI, Hoischen A, Hoyng C, Klevering BJ, van den Born LI, Veltman JA, Cremers FPM, Scheffer H: Next-generation genetic testing for retinitis pigmentosa. Human Mutation 2012, 33:963-72.
[8] Graziotto JJ, Farkas MH, Bujakowska K, Deramaudt BM, Zhang Q, Nandrot EF, Inglehearn CF, Bhattacharya SS, Pierce EA: Three gene-targeted mouse models of RNA splicing factor RP show late-onset RPE and retinal degeneration. Investigative Ophthalmology & Visual Science 2011, 52:190-8.
[9] Graziotto JJ, Inglehearn CF, Pack MA, Pierce EA: Decreased Levels of the RNA Splicing Factor Prpf3 in Mice and Zebrafish Do Not Cause Photoreceptor Degeneration. Investigative Ophthalmology & Visual Science 2008, 49:3830-8.
[10] Rio Frio T, Wade NM, Ransijn A, Berson EL, Beckmann JS, Rivolta C: Premature termination codons in PRPF31 cause retinitis pigmentosa via haploinsufficiency due to nonsense-mediated mRNA decay. The Journal of Clinical Investigation 2008, 118:1519-31.
[11] Venturini G, Rose AM, Shah AZ, Bhattacharya SS, Rivolta C: CNOT3 is a modifier of PRPF31 mutations in retinitis pigmentosa with incomplete penetrance. PLoS Genetics 2012, 8.
[12] Kevany BM, Palczewski K: Phagocytosis of Retinal Rod and Cone Photoreceptors. Physiology 2010, 25:8-15.
[13] Nandrot EF, Kim Y, Brodie SE, Huang X, Sheppard D, Finnemann SC: Loss of synchronized retinal phagocytosis and age-related blindness in mice lacking alphavbeta5 integrin. The Journal of Experimental Medicine 2004, 200:1539-45.
[14] Nandrot EF, Anand M, Sircar M, Finnemann SC: Novel role for alphavbeta5-integrin in retinal adhesion and its diurnal peak. American Journal of Physiology Cell Physiology 2006, 290(4):C1256-62.
[15] Nandrot EF, Anand M, Almeida D, Atabai K, Sheppard D, Finnemann SC: Essential role for MFG-E8 as ligand for alphavbeta5 integrin in diurnal retinal phagocytosis. Proceedings of the National Academy of Sciences of the United States of America 2007, 104:12005-10.
[16] Ruggiero L, Connor MP, Chen J, Langen R, Finnemann SC: Diurnal, localized exposure of phosphatidylserine by rod outer segment tips in wild-type but not Itgb5−/− or Mfge8−/− mouse retina. Proceedings of the National Academy of Sciences 2012, 109:8145-8.
[17] Nandrot EF, Finnemann SC: Lack of alphavbeta5 integrin receptor or its ligand MFG-E8: distinct effects on retinal function. Ophthalmic Research 2008, 40:120-3.
[18] Nandrot E, Dufour EM, Provost AC, Péquignot MO, Bonnel S, Gogat K, Marchant D, Rouillac C, Sépulchre de Condé B, Bihoreau M-T: Homozygous Deletion in the Coding Sequence of the c-mer Gene in RCS Rats Unravels General Mechanisms of Physiological Cell Adhesion and Apoptosis. Neurobiology of Disease 2000, 7:586-99.
[19] Issa PC, Bolz HJ, Ebermann I, Domeier E, Holz FG, Scholl HP: Characterisation of severe rod-cone dystrophy in a consanguineous family with a splice site mutation in the MERTK gene. British Journal of Ophthalmology 2009, 93:920-5.
[20] Ostergaard E, Duno M, Batbayli M, Vilhelmsen K, Rosenberg T: A novel MERTK deletion is a common founder mutation in the Faroe Islands and is responsible for a high proportion of retinitis pigmentosa cases. Molecular Vision 2011, 17:1485.
[21] Davies JQ and Gordon S: Isolation and culture of murine macrophages. Basic cell culture protocols 2005: 91-103.
[22] Stenson PD, Mort M, Ball EV, Shaw K, Phillips AD, Cooper DN: The Human Gene Mutation Database: building a comprehensive mutation repository for clinical and molecular genetics, diagnostic testing and personalized genomic medicine. Human Genetics 2013: 1-9.
[23] Mao Y, Finnemann S: Analysis of Photoreceptor Outer Segment Phagocytosis by RPE Cells in Culture. Retinal Degeneration. Edited by Weber BHF, Langmann T. Humana Press, 2013. pp. 285-95.
[24] Marmorstein AD: The Polarity of the Retinal Pigment Epithelium. Traffic 2001, 2:867-72.
[25] Davidson AE, Millar ID, Urquhart JE, Burgess-Mullan R, Shweikh Y, Parry N, O'Sullivan J, Maher GJ, Mckibbin M, Downes SM, Lotery AJ, Jacobson SG, Brown PD, Black GC, Manson FD: Missense mutations in a retinal pigment epithelium protein, bestrophin-1, cause retinitis pigmentosa. American journal of Human Genetics 2009, 85:581-92.
[26] Lopes VS, Gibbs D, Libby RT, Aleman TS, Welch DL, Lillo C, Jacobson SG, Radu RA, Steel KP, Williams DS: The Usher 1B protein, MYO7A, is required for normal localization and function of the visual retinoid cycle enzyme, RPE65. Human Molecular Genetics 2011, 20:2560-70.
[27] Finnemann SC, Bonilha VL, Marmorstein AD, Rodriguez-Boulan E: Phagocytosis of rod outer segments by retinal pigment epithelial cells requires αvβ5 integrin for binding but not for internalization. Proceedings of the National Academy of Sciences 1997, 94:12932-7.
[28] Prasad D, Rothlin CV, Burrola P, Burstyn-Cohen T, Lu Q, Garcia de Frutos P, Lemke G: TAM receptor function in the retinal pigment epithelium. Molecular and Cellular Neuroscience 2006, 33:96-108.
[29] Finnemann SC: Focal adhesion kinase signaling promotes phagocytosis of integrin-bound photoreceptors. The EMBO Journal 2003, 22:4143-54.
[30] Qin S, Rodrigues GA: Roles of alphavbeta5, FAK and MerTK in oxidative stress inhibition of RPE cell phagocytosis. Experimental Eye Research 2012, 94:63-70.
[31] Nandrot EF, Silva KE, Scelfo C, Finnemann SC: Retinal pigment epithelial cells use a MerTK-dependent mechanism to limit the phagocytic particle binding activity of alphavbeta5 integrin. Biology of the cell/under the auspices of the European Cell Biology Organization 2012, 104:326-41.
[32] Hall MO, Obin MS, Heeb MJ, Burgess BL, Abrams TA: Both protein S and Gas6 stimulate outer segment phagocytosis by cultured rat retinal pigment epithelial cells. Experimental Eye Research 2005, 81:581-91.
[33] Burstyn-Cohen T, Lew ED, Través PG, Burrola PG, Hash JC, Lemke G: Genetic Dissection of TAM Receptor-Ligand Interaction in Retinal Pigment Epithelial Cell Phagocytosis. Neuron 2012, 76:1123-32.
[34] Yin J, Brocher J, Fischer U, Winkler C: Mutant Prpf31 causes pre-mRNA splicing defects and rod photoreceptor cell degeneration in a zebrafish model for Retinitis pigmentosa. Molecular Neurodegeneration 2011, 6:1-18.
[35] Masland RH: Cell populations of the retina: the Proctor lecture. Investigative Ophthalmology & Visual Science 2011, 52:4581-91.
[36] Finnemann SC, Nandrot EF: MerTK activation during RPE phagocytosis in vivo requires alpha Vbeta5 integrin. Advances in Experimental Medicine and Biology 2006, 572:499-503.
[37] Gal A, Li Y, Thompson DA, Weir J, Orth U, Jacobson SG, Apfelstedt-Sylla E, Vollrath D: Mutations in MERTK, the human orthologue of the RCS rat retinal dystrophy gene, cause retinitis pigmentosa. Nature Genetics 2000, 26:270-1.
[38] Mackay DS, Henderson RH, Sergouniotis PI, Li Z, Moradi P, Holder GE, Waseem N, Bhattacharya SS, Aldahmesh MA, Alkuraya FS: Novel mutations in MERTK associated with childhood onset rod-cone dystrophy. Molecular Vision 2010, 16:369-377.
[39] Tschernutter M, Jenkins S, Waseem N, Saihan Z, Holder G, Bird A, Bhattacharya S, Ali R, Webster A: Clinical characterisation of a family with retinal dystrophy caused by mutation in the Mertk gene. British Journal of Ophthalmology 2006, 90:718-23.
[40] Taniguchi-Ikeda M, Kobayashi K, Kanagawa M, Yu C-c, Mori K, Oda T, Kuga A, Kurahashi H, Akman HO, DiMauro S: Pathogenic exon-trapping by SVA retrotransposon and rescue in Fukuyama muscular dystrophy. Nature 2011, 478:127-31.
[41] Dell'Angelica EC: AP-3-dependent trafficking and disease: the first decade. Current Opinion in Cell Biology 2009, 21:552-9.
[42] Farkas MH, Grant GR, White JA, Sousa ME, Consugar MB, Pierce EA: Transcriptome analyses of the human retina identify unprecedented transcript diversity and 3.5 Mb of novel transcribed sequence via significant alternative splicing and novel genes. BMC Genomics 2013, 14:486.
[43] Buchholz DE, Pennington BO, Croze RH, Hinman CR, Coffey PJ, Clegg DO: Rapid and Efficient Directed Differentiation of Human Pluripotent Stem Cells Into Retinal Pigmented Epithelium. Stem Cells Translational Medicine 2013, 2:384-93.
[44] Meyer JS, Howden SE, Wallace KA, Verhoeven AD, Wright LS, Capowski EE, Pinilla I, Martin JM, Tian S, Stewart R: Optic Vesicle-like Structures Derived from Human Pluripotent Stem Cells Facilitate a Customized Approach to Retinal Disease Treatment. Stem Cells 2011, 29:1206-18.
[45] Singh R, Phillips MJ, Kuai D, Meyer J, Martin JM, Smith MA, Perez ET, Shen W, Wallace KA, Capowski EE: Functional analysis of serially expanded human iPS cell-derived RPE cultures. Investigative Ophthalmology & Visual Science 2013, 54:6767-78.

### Example 2. Development and Functional Characterization of PRPF31 Knockout ARPE-19 Cells Using Genome Editing Techniques

As described in Example 1, retinal pigment epithelium (RPE) was identified as the site of pathogenesis in three mutant mouse models of RNA splicing retinitis pigmentosa (RP). However, these results needed to be confirmed in human RPE. With the advent of CRISPR/Cas9 genome editing techniques, human cell line models were developed for these forms of disease. This example presents the use of CRISPR/Cas9 genome editing to knockout PRPF31 for the first time in human cell lines and characterize the effect on RPE function.

A 20 bp guide RNA (gRNA) to exon 7 of PRPF31 was designed and cloned into a pCAG vector containing gRNA scaffolding sequence. The gRNA vector was co-transfected with a pCAG-Cas9-GFP vector into ARPE-19 cells.

GFP positive cells were single cell sorted into individual wells of a 96 well plate and grown to confluence. DNA was isolated from each clone and the region around the predicted cut site was Sanger sequenced to identify those that exhibit correct cutting and non-homologous end joining (NHEJ). Five NHEJ lines were selected for further characterization using both qRT-PCR and phagocytosis assays to quantify FITC-labeled photoreceptor outer segment uptake with flow cytometry. These lines were maintained as confluent cultures for 3 weeks to ensure polarization and maximal expression of RPE-specific genes.

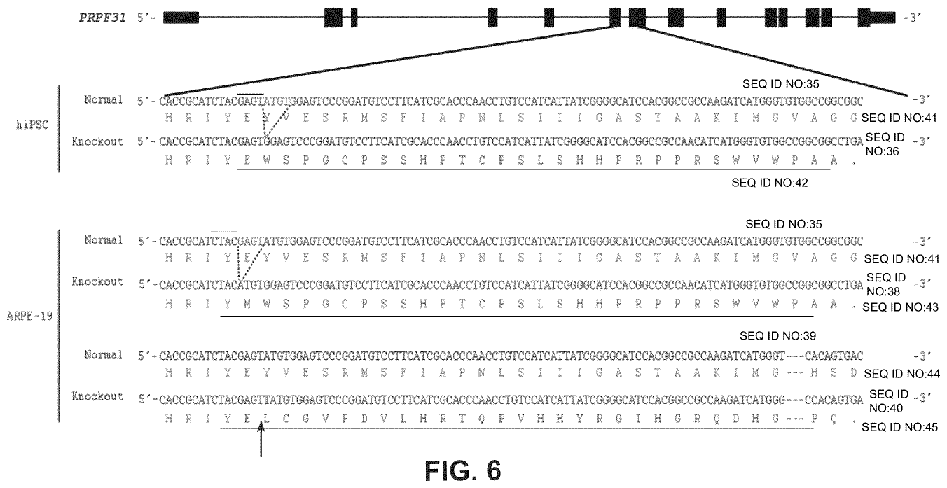

Approximately 25% of the individual clones validated following transfection showed NHEJ with deletions between 2 and 11 bases and one clone had a 1 base insertion (FIG. 6). Only heterozygous indels were identified, consistent with previous reports that mutations in PRPF31 cause disease via haploinsufficiency. Expression of PRPF31 in 4 of the 5 genome edited clones was significantly (P<0.05) reduced by 50-80%, as compared to the wild-type control. To confirm these changes were a result of genome editing, expression levels of the PRPF31 modifier CNOT3 were determined. One line had a 2-fold increase in expression, which may explain reduced levels of PRPF31 in that line. Flow cytometry analysis of POS uptake demonstrated phagocytosis was reduced by 10-60-fold in the genome edited lines.

Currently, it is difficult to study the disease mechanism of RNA splicing factor RP in human models. We have created a human cell line model for PRPF31-associated disease that mimics findings in mouse models. These lines will allow us to study the disease in a more relevant model, affording us the capability to interrogate splicing more deeply. Further, we can study the effect of AAV-mediated gene augmentation of PRPF31 on disease pathogenesis and rescue of functional deficiencies.

For example, as noted in Example 1, photoreceptor outer segments (POS) are completely renewed every 10 days by continuous growth at their bases regulated by shedding of discs at their distal tips (Young R W. The renewal of photoreceptor cell outer segments. Journal of Cell Biology. 1967; 33:61-72; Young RW. Shedding of discs from rod outer segments in the rhesus monkey. JUltrastructRes. 1971; 34:190-203). Phagocytosis of the spent POS material by the RPE is essential for proper retinal function, as its absence or delay leads to loss of vision (Dowling J E, Sidman R L. Inherited retinal dystrophy in the rat. Journal Cell Biology. 1962; 14:73-109;Nandrot E F, Kim Y, Brodie S E, Huang X, Sheppard D, Finnemann S C. Loss of synchronized retinal phagocytosis and age-related blindness in mice lacking alphavbeta5 integrin. Journal Experimental Medicine. 2004; 200:1539-45). Phagocytosis of POS was decreased in primary RPE cell cultures from 10-day old mutant mice, and this was replicated by shRNA-mediated knockdown of PRPF31 in human ARPE-19 cells (Example 1, FIG. 1). The diurnal rhythmicity of phagocytosis in vivo was also lost, and the strength of the adhesion between RPE apical microvilli and POS declined at the time of peak adhesion in all 3 mutant models (Nandrot E F, Kim Y, Brodie S E, Huang X, Sheppard D, Finnemann S C. Loss of synchronized retinal phagocytosis and age-related blindness in mice lacking alphavbeta5 integrin. Journal Experimental Medicine. 2004; 200:1539-45; Nandrot E F, Finnemann S C. Lack of alphavbeta5 integrin receptor or its ligand MFG-E8: distinct effects on retinal function. Ophthalmic Research. 2008; 40:120-3).

### Example 3. Development and Functional Characterization of PRPF31 Knockout Human Induced Pluriopotent Stem Cells (hiPSCs) Using Genome Editing Techniques

CRISPR/Cas9 genome editing was used to knockout PRPF31 in normal hiPSCs (Hou Z, Zhang Y, Propson N E, Howden S E, Chu L F, Sontheimer E J, Thomson J A. Efficient genome engineering in human pluripotent stem cells using Cas9 from Neisseria meningitidis. Proceedings of the National Academy of Sciences of the United States of America. 2013; 110:15644-9; Xue H, Wu J, Li S, Rao M S, Liu Y. Genetic Modification in Human Pluripotent Stem Cells by Homologous Recombination and CRISPR/Cas9 System. Methods Molecular Biology. 2014 Peters D T, Cowan C A, Musunuru K. Genome editing in human pluripotent stem cells. StemBook. Cambridge (MA) 2013; Ding Q, Regan S N, Xia Y, Oostrom L A, Cowan C A, Musunuru K. Enhanced efficiency of human pluripotent stem cell genome editing through replacing TALENs with CRISPRs. Cell Stem Cell. 2013; 12:393-4. PMCID: 3925309) as described above in Example 2.

The development of hiPSC technology now makes it possible to determine if human RPE cells are similarly affected by mutations in RNA splicing factor genes, since hiPSCs can be readily differentiated into RPE cells (see Example 1, Singh R, Phillips M J, Kuai D, Meyer J T, Martin J, Smith M, Shen W, Perez E T, Wallace K A, Capowski E E, Wright L, Gamm D M. Functional analysis of serially expanded human iPS cell-derived RPE cultures. Investigative Ophthalmology & Visual Science. 2013; 54:6767-78; Buchholz D E, Hikita S T, Rowland T J, Friedrich A M, Hinman C R, Johnson L V, Clegg D O. Derivation of functional retinal pigmented epithelium from induced pluripotent stem cells. Stem Cells. 2009; 27:2427-34; Okamoto S, Takahashi M. Induction of retinal pigment epithelial cells from monkey iPS cells. Investigative Ophthalmology & Visual Science. 2011; 52:8785-90; Ukrohne T U, Westenskow P D, Kurihara T, Friedlander D F, Lehmann M, Dorsey A L, Li W, Zhu S, Schultz A, Wang J, Siuzdak G, Ding S, Friedlander M. Generation of retinal pigment epithelial cells from small molecules and OCT4 reprogrammed human induced pluripotent stem cells. Stem cells translational medicine. 2012; 1:96-109; Westenskow P D, Moreno S K, Krohne T U, Kurihara T, Zhu S, Zhang Z N, Zhao T, Xu Y, Ding S, Friedlander M. Using flow cytometry to compare the dynamics of photoreceptor outer segment phagocytosis in iPS-derived RPE cells. Investigative Ophthalmology & Visual Science. 2012; 53:6282-90; Buchholz D E, Pennington B O, Croze R H, Hinman C R, Coffey P J, Clegg D O. Rapid and efficient directed differentiation of human pluripotent stem cells into retinal pigmented epithelium. Stem cells translational medicine. 2013; 2:384-93). hiPSC-derived RPE cells share many features with native RPE cells, including functional tight junctions, phagocytosis of POS, and polarization (Ibid).

To obtain hiPSC-RPE, embryoid bodies (EBs) are generated, adhered to laminin-coated plates and cultured in retinal differentiation medium (RDM) for 60-90 days. Regions of pigmented cells will then be microdissected, dissociated and passed onto transwell inserts according to established protocols (Singh R, Phillips M J, Kuai D, Meyer J T, Martin J, Smith M, Shen W, Perez E T, Wallace K A, Capowski E E, Wright L, Gamm D M. Functional analysis of serially expanded human iPS cell-derived RPE cultures. Investigative Ophthalmology & Visual Science. 2013; 54:6767-78; Phillips M J, Wallace K A, Dickerson S J, Miller M J, Verhoeven A D, Martin J M, Wright L S, Shen W, Capowski E E, Percin E F, Perez E T, Zhong X, Canto-Soler M V, Gamm D M. Blood-derived human iPS cells generate optic vesicle-like structures with the capacity to form retinal laminae and develop synapses. Investigative Ophthalmology & Visual Science. 2012; 53:2007-19). Cells will then be cultured for an additional 30-60 days, when pigmented monolayers reform. Prior to use in experiments the transepithelial resistance (TER) of the hiPSC-RPE monolayers grown on Transwell inserts will be measured; only those cultured with TER>150 Ωcm² will be selected for further study. For every experiment, we will include duplicates for each mutation of interest and wild-type control cells, which will be cultured and analyzed in parallel. The structure and function of the hiPSC-derived RPE cells will be characterized using several methods:

Structure. Light microscopy and electron microscopy is used to assess polarization, including formation of apical processes and basolateral infoldings (Exaple 1, Garland D L, Fernandez-Godino R, Kaur I, Speicher K D, Harnly J M, Lambris J D, Speicher D W, Pierce E A. Mouse genetics and proteomic analyses demonstrate a critical role for complement in a model of DHRD/ML, an inherited macular degeneration. Human Molecular Genetics. 2013; September 4. [Epub ahead of print]; Liu Q, Lyubarsky A, Skalet J H, Pugh E N, Jr., Pierce E A. RP1 is required for the correct stacking of outer segment discs. Investigative Ophthalmology & Visual Science. 2003; 44:4171-83).

Phagocytosis. As described above, primary cultures of RPE cells from the Prpf3^{T494M/T494M}, Prpf8^{H2309P/H2309P}, and Prpf31^{+/-} mice have significantly decreased ability to phagocytose POS (FIG. 1). We will assess the phagocytic function of hiPSC-derived RPE cells using established techniques (see Example 1; Finnemann S C, Bonilha V L, Marmorstein A D, Rodriguez-Boulan E. Phagocytosis of rod outer segments by retinal pigment epithelial cells requires alpha(v)beta5 integrin for binding but not for internalization. Proc Natl Acad Sci USA. 1997; 94:12932-7; Singh R, Shen W, Kuai D, Martin J M, Guo X, Smith M A, Perez E T, Phillips M J, Simonett J M, Wallace K A, Verhoeven A D, Capowski E E, Zhang X, Yin Y, Halbach P J, Fishman G A, Wright L S, Pattnaik B R, Gamm D M. iPS cell modeling of Best disease: insights into the pathophysiology of an inherited macular degeneration. Human Molecular Genetics. 2013; 22:593-60.)

To assess the polarity of the hiPSC-derived RPE cells, vibratome sections of stably transfected cells grown on Transwells are immunostained with antibodies against established RPE cell markers using established techniques (Nandrot E F, Kim Y, Brodie S E, Huang X, Sheppard D, Finnemann S C. Loss of synchronized retinal phagocytosis and age-related blindness in mice lacking alphavbeta5 integrin. Journal Experimental Medicine. 2004; 200:1539-45; Nandrot E F, Finnemann S C. Lack of alphavbeta5 integrin receptor or its ligand MFG-E8: distinct effects on retinal function. Ophthalmic Research. 2008; 40:120-3; Finnemann S C, Nandrot E F. MerTK activation during RPE phagocytosis in vivo requires alpha Vbeta5 integrin. Advances Experimental Medicine Biology. 2006; 572:499-503). The stained cells will be evaluated by confocal microscopy, and the distribution and relative amounts of the marker proteins will be compared in mutant and control hiPSC-derived RPE cells. The levels of these RPE cell markers will also be evaluated in differentiated cells via western blotting (Nandrot E F, Kim Y, Brodie S E, Huang X, Sheppard D, Finnemann S C. Loss of synchronized retinal phagocytosis and age-related blindness in mice lacking alphavbeta5 integrin. Journal Experimental Medicine. 2004; 200:1539-45).

Changes in RPE phenotype observed in the genome edited hiPSCs are confirmed using hiPSCs from patients with RNA splicing factor RP. Patients and families with RP due to mutations in the PRPF31 gene have been identified, and hiPSCs are generated using fibroblasts from one affected and one unaffected family member from each of 3 families. Briefly, fibroblasts are reprogrammed using non-integrating, oriP-containing plasmid vectors encoding seven reprogramming factors (OCT4, SOX2, NANOG, LIN28, c-Myc, KLF4, and SV40 large T-antigen), as described (Yu J, Hu K, Smuga-Otto K, Tian S, Stewart R, Slukvin I I, Thomson J A. Human induced pluripotent stem cells free of vector and transgene sequences. Science. 2009; 324:797-801). hiPSC lines with normal karyotypes and that are confirmed to be pluripotent by teratoma studies and expression of the pluripotency markers OCT4, SSEA4, NANOG and TRA-1-81 would be selected for further study (Singh R, Shen W, Kuai D, Martin J M, Guo X, Smith M A, Perez E T, Phillips M J, Simonett J M, Wallace K A, Verhoeven A D, Capowski E E, Zhang X, Yin Y, Halbach P J, Fishman G A, Wright L S, Pattnaik B R, Gamm D M. iPS cell modeling of Best disease: insights into the pathophysiology of an inherited macular degeneration. Human Molecular Genetics. 2013; 22:593-607; Singh R, Phillips M J, Kuai D, Meyer J T, Martin J, Smith M, Shen W, Perez E T, Wallace K A, Capowski E E, Wright L, Gamm D M. Functional analysis of serially expanded human iPS cell-derived RPE cultures. Investigative Ophthalmology & Visual Science. 2013; 54:6767-78; Meyer J S, Howden S E, Wallace K A, Verhoeven A D, Wright L S, Capowski E E, Pinilla I, Martin J M, Tian S, Stewart R, Pattnaik B, Thomson J A, Gamm D M. Optic vesicle-like structures derived from human pluripotent stem cells facilitate a customized approach to retinal disease treatment. Stem Cells. 2011; 29:1206-18). After confirming that each hiPSC line carries the expected mutation, hiPSC-derived RPE function is characterized in cells from patients and compared to unaffected family members using the techniques described above.

### Example 4. AAV Vectors for Gene Augmentation Therapy

The identification of RPE cells as likely to be the primary cells affected in RNA splicing factor RP (see Example 1) creates an opportunity to use gene augmentation therapy for diseases caused by mutations in PRPF31. To achieve this goal, we have developed AAV vectors for expressing human PRPF31 in RPE cells, and tested the ability of the AAV-delivered PRPF31 to ameliorate the phenotype in cultured RPE cells, and then in Prpf31^{+/-} mice in vivo. AAV is the preferred gene delivery vector for retinal disorders based on the success of clinical trials of gene therapy for RPE65 LCA and choroideremia, as well as other clinical and pre-clinical studies (Maguire A M, Simonelli F, Pierce E A, Pugh E N, Jr., Mingozzi F, Bennicelli J, Banfi S, Marshall K A, Testa F, Surace E M, Rossi S, Lyubarsky A, Arruda V R, Konkle B, Stone E, Sun J, Jacobs J, Dell'Osso L, Hertle R, Ma J X, Redmond T M, Zhu X, Hauck B, Zelenaia O, Shindler K S, Maguire M G, Wright J F, Volpe N J, McDonnell J W, Auricchio A, High K A, Bennett J. Safety and efficacy of gene transfer for Leber's congenital amaurosis. New England Journal of Medicine. 2008; 358:2240-8. PMCID: 2829748; Bainbridge J W, Smith A J, Barker S S, Robbie S, Henderson R, Balaggan K, Viswanathan A, Holder G E, Stockman A, Tyler N, Petersen-Jones S, Bhattacharya S S, Thrasher A J, Fitzke F W, Carter B J, Rubin G S, Moore A T, Ali R R. Effect of gene therapy on visual function in Leber's congenital amaurosis. New England Journal of Medicine. 2008; 358:2231-9; Cideciyan A V, Aleman T S, Boye S L, Schwartz S B, Kaushal S, Roman A J, Pang J J, Sumaroka A, Windsor E A, Wilson J M, Flotte T R, Fishman G A, Heon E, Stone E M, Byrne B J, Jacobson S G, Hauswirth W W. Human gene therapy for RPE65 isomerase deficiency activates the retinoid cycle of vision but with slow rod kinetics. Proceedings National Academy Sciences USA. 2008; 105:15112-7. PMCID: 2567501; Maguire A M, High K A, Auricchio A, Wright J F, Pierce E A, Testa F, Mingozzi F, Bennicelli J L, Ying G S, Rossi S, Fulton A, Marshall K A, Banfi S, Chung D C, Morgan J I, Hauck B, Zelenaia O, Zhu X, Raffini L, Coppieters F, De Baere E, Shindler K S, Volpe N J, Surace E M, Acerra C, Lyubarsky A, Redmond T M, Stone E, Sun J, McDonnell J W, Leroy B P, Simonelli F, Bennett J. Age-dependent effects of RPE65 gene therapy for Leber's congenital amaurosis: a phase 1 dose-escalation trial. Lancet. 2009; 374:1597-605; Jacobson S G, Cideciyan A V, Ratnakaram R, Heon E, Schwartz S B, Roman A J, Peden M C, Aleman T S, Boye S L, Sumaroka A, Conlon T J, Calcedo R, Pang J J, Erger K E, Olivares M B, Mullins C L, Swider M, Kaushal S, Feuer W J, Iannaccone A, Fishman G A, Stone E M, Byrne B J, Hauswirth W W. Gene therapy for leber congenital amaurosis caused by RPE65 mutations: safety and efficacy in 15 children and adults followed up to 3 years. Archives Ophthalmology. 2012; 130:9-24; Bennett J, Ashtari M, Wellman J, Marshall K A, Cyckowski L L, Chung D C, McCague S, Pierce E A, Chen Y, Bennicelli J L, Zhu X, Ying G S, Sun J, Wright J F, Auricchio A, Simonelli F, Shindler K S, Mingozzi F, High K A, Maguire A M. AAV2 gene therapy readministration in three adults with congenital blindness. Science translational medicine. 2012; 4:120ra15; Bowles D E, McPhee S W, Li C, Gray S J, Samulski J J, Camp A S, Li J, Wang B, Monahan P E, Rabinowitz J E, Grieger J C, Govindasamy L, Agbandje-McKenna M, Xiao X, Samulski R J. Phase 1 gene therapy for Duchenne muscular dystrophy using a translational optimized AAV vector. Molecular therapy: the journal of the American Society of Gene Therapy. 2012; 20:443-55. PMCID: 3277234; Maclachlan T K, Lukason M, Collins M, Munger R, Isenberger E, Rogers C, Malatos S, Dufresne E, Morris J, Calcedo R, Veres G, Scaria A, Andrews L, Wadsworth S. Preclinical safety evaluation of AAV2-sFLT01-a gene therapy for age-related macular degeneration. Molecular Therapy. 2011; 19:326-34. PMCID: 3034852; Nathwani A C, Tuddenham E G, Rangarajan S, Rosales C, McIntosh J, Linch D C, Chowdary P, Riddell A, Pie A J, Harrington C, O'Beirne J, Smith K, Pasi J, Glader B, Rustagi P, Ng C Y, Kay M A, Zhou J, Spence Y, Morton C L, Allay J, Coleman J, Sleep S, Cunningham J M, Srivastava D, Basner-Tschakarjan E, Mingozzi F, High K A, Gray J T, Reiss U M, Nienhuis A W, Davidoff A M. Adenovirus-associated virus vector-mediated gene transfer in hemophilia B. New England Journal of Medicine. 2011; 365:2357-65. PMCID: 3265081).

AAV has an exceptional safety record in early phase clinical studies and also poses less risk of genotoxicity compared to other vector systems since AAV genomes are stable in an episomal form in terminally differentiated cells such as photoreceptor and RPE cells (Yang G S, Schmidt M, Yan Z, Lindbloom J D, Harding T C, Donahue B A, Engelhardt J F, Kotin R, Davidson B L. Virus-mediated transduction of murine retina with adeno-associated virus: effects of viral capsid and genome size. J Virol. 2002; 76:7651-60; Acland G M, Aguirre G D, Bennett J, Aleman T S, Cideciyan A V, Bennicelli J, Dejneka N S, Pearce-Kelling S E, Maguire A M, Palczewski K, Hauswirth W W, Jacobson S G. Long-term restoration of rod and cone vision by single dose rAAV-mediated gene transfer to the retina in a canine model of childhood blindness. Molecular Therapy. 2005; 12:1072-82).

Several AAV vector systems for PFPF31 are developed, with different promoter choices and capsid serotypes. With regard to promoters, vectors can include promoters that drive expression in many cell types (e.g., CAG or CASI), RPE cells (e.g., promotors for RPE-specific proteins such as VMD2, RPE65, RLBP1, RGR, or TIMP3) and photoreceptor cells (RHO) (Esumi N, Oshima Y, Li Y, Campochiaro P A, Zack D J. Analysis of the VMD2 promoter and implication of E-box binding factors in its regulation. Journal Biological Chemistry. 2004; 279:19064-73; Guziewicz K E, Zangerl B, Komaromy A M, Iwabe S, Chiodo V A, Boye S L, Hauswirth W W, Beltran W A, Aguirre G D. Recombinant AAV-Mediated BEST1 Transfer to the Retinal Pigment Epithelium: Analysis of Serotype-Dependent Retinal Effects. PLoS One. 2013; 8:e75666; Allocca M, Mussolino C, Garcia-Hoyos M, Sanges D, Iodice C, Petrillo M, Vandenberghe L H, Wilson J M, Marigo V, Surace E M, Auricchio A. Novel adeno-associated virus serotypes efficiently transduce murine photoreceptors. J Virol. 2007; 81:11372-80). The components of the AAV vectors are synthesized using codon-optimized PRPF31 sequences to improve the level and duration of gene expression (Ill C R, Chiou H C. Gene therapy progress and prospects: recent progress in transgene and RNAi expression cassettes. Gene Therapy. 2005; 12:795-802; Foster H, Sharp P S, Athanasopoulos T, Trollet C, Graham I R, Foster K, Wells D J, Dickson G. Codon and mRNA sequence optimization of microdystrophin transgenes improves expression and physiological outcome in dystrophic mdx mice following AAV2/8 gene transfer. Molecular Therapy. 2008; 16:1825-32; Sack B K, Merchant S, Markusic D M, Nathwani A C, Davidoff A M, Byrne B J, Herzog R W. Transient B cell depletion or improved transgene expression by codon optimization promote tolerance to factor VIII in gene therapy. PLoS One. 2012; 7:e37671). In preliminary studies, codon optimized PRPF31 produced full-length PRPF31 protein in ARPE-19 cells. The vectors prepared encode minimal vector genome necessary to achieve optimal expression. Since we are interested primarily in transducing RPE cells, we will use AAV2 as a control serotype, as this vector is known to transduce cultured monolayer cells and transduced the RPE well in vivo (Pang J J, Lauramore A, Deng W T, Li Q, Doyle T J, Chiodo V, Li J, Hauswirth W W. Comparative analysis of in vivo and in vitro AAV vector transduction in the neonatal mouse retina: effects of serotype and site of administration. Vision Research. 2008; 48:377-85; Vandenberghe L H, Bell P, Maguire A M, Cearley C N, Xiao R, Calcedo R, Wang L, Castle M J, Maguire A C, Grant R, Wolfe J H, Wilson J M, Bennett J. Dosage thresholds for AAV2 and AAV8 photoreceptor gene therapy in monkey. Science translational medicine. 2011; 3:88ra54; Tolmachova T, Tolmachov O E, Barnard A R, de Silva S R, Lipinski D M, Walker N J, Maclaren R E, Seabra M C. Functional expression of Rab escort protein 1 following AAV2-mediated gene delivery in the retina of choroideremia mice and human cells ex vivo. Journal of Molecular Medicine. 2013; 91:825-37. PMCID: 3695676). Vector preparations are generated and purified using established techniques (Vandenberghe L H, Bell P, Maguire A M, Cearley C N, Xiao R, Calcedo R, Wang L, Castle M J, Maguire A C, Grant R, Wolfe J H, Wilson J M, Bennett J. Dosage thresholds for AAV2 and AAV8 photoreceptor gene therapy in monkey. Science translational medicine. 2011; 3:88ra54, Lock M, Alvira M, Vandenberghe L H, Samanta A, Toelen J, Debyser Z, Wilson J M. Rapid, simple, and versatile manufacturing of recombinant adeno-associated viral vectors at scale. Human Gene Therapy. 2010; 21:1259-71. PMCID: 2957274). Titration is performed by Taqman qPCR with primer-probe sets directed toward the poly-adenylation signal in the vector genome.

To study PRPF31 expression in cultured cells, the PRPF31 mutant and control ARPE-19 cells are cultured on Transwell filters, as described in Example 1. Cells are treated with the desired amount of AAV-PRPF31 vectors, and cultured for an additional 11-14 days. Wild-type RPE cells treated with AAV-PRPF31, and PRFP31^{+/-} cells treated with AAV-EGFP are used as controls. The effects of the AAV-PRPF31 treatment are evaluated using several approaches. The production of full-length PRPF31 protein is evaluated by immunofluorescence microscopy and western blotting experiments 2-4 days following transduction (Liu Q, Zhou J, Daiger S P, Farber D B, Heckenlively J R, Smith J E, Sullivan L S, Zuo J, Milam A H, Pierce E A. Identification and subcellular localization of the RP1 protein in human and mouse photoreceptors. Investigative Ophthalmology & Visual Science. 2002; 43:22-32; Liu Q, Zuo J, Pierce E A. The retinitis pigmentosa 1 protein is a photoreceptor microtubule-associated protein. Journal Neuroscience. 2004; 24:6427-36; Falk M J, Zhang Q, Nakamaru-Ogiso E, Kannabiran C, Fonseca-Kelly Z, Chakarova C, Audo I, Mackay D S, Zeitz C, Borman A D, Staniszewska M, Shukla R, Palavalli L, Mohand-Said S, Waseem N H, Jalali S, Perin J C, Place E, Ostrovsky J, Xiao R, Bhattacharya S S, Consugar M, Webster A R, Sahel J A, Moore A T, Berson E L, Liu Q, Gai X, Pierce E A. NMNAT1 mutations cause Leber congenital amaurosis. Nature Genetics. 2012; 44:1040-5). Gene transfer is assayed by qPCR for vector genomes. Restoration of the normal phagocytic activity of the mutant cells is measured by treatment with FITC-labeled POS, using established techniques (Example 1, Finnemann S C, Bonilha V L, Marmorstein A D, Rodriguez-Boulan E. Phagocytosis of rod outer segments by retinal pigment epithelial cells requires alpha(v)beta5 integrin for binding but not for internalization. Proc Natl Acad Sci USA. 1997; 94:12932-7; Singh R, Shen W, Kuai D, Martin J M, Guo X, Smith M A, Perez E T, Phillips M J, Simonett J M, Wallace K A, Verhoeven A D, Capowski E E, Zhang X, Yin Y, Halbach P J, Fishman G A, Wright L S, Pattnaik B R, Gamm D M. iPS cell modeling of Best disease: insights into the pathophysiology of an inherited macular degeneration. Human Molecular Genetics. 2013; 22:593-607).

To study PRPF31 expression and function in Prpf31^{+/-} mutant mice, AAV-mediated delivery of PRPF31 is used to treat the defective phagocytosis in Prpf31^{+/-} mice in vivo. For these studies, the optimal doses of the AAV-PRPF31 vectors identified in cell culture studies is injected sub-retinally into one eye of Prpf31^{+/-} mice. Eyes are harvested 1 month after injection and evaluated for expression and localization of the full-length PRPF31 protein using immunofluorescence and western blotting assays (Liu Q, Lyubarsky A, Skalet J H, Pugh E N, Jr., Pierce E A. RP1 is required for the correct stacking of outer segment discs. Investigative Ophthalmology & Visual Science. 2003; 44:4171-83; Liu Q, Saveliev A, Pierce E A. The severity of retinal degeneration in Rp1h gene-targeted mice is dependent on genetic background. Investigative Ophthalmology & Visual Science. 2009; 50:1566-74; Liu Q, Collin R W, Cremers F P, den Hollander A I, van den Born L I, Pierce E A. Expression of Wild-Type Rp1 Protein in Rp1 Knock-in Mice Rescues the Retinal Degeneration Phenotype. PLoS One. 2012; 7:e43251).

The ability of AAV-delivered PRPF31 to prevent and/or rescue the loss of rhythmicity of RPE phagocytosis is assessed at 2 hours before light onset (−2), at light onset (0), and 2, 4, and 6 (+2, +4, +6) hours after light onset using established techniques for immunofluorescent staining for rhodopsin and detection of phagosomes located in the RPE cell layer (Nandrot E F, Kim Y, Brodie S E, Huang X, Sheppard D, Finnemann S C. Loss of synchronized retinal phagocytosis and age-related blindness in mice lacking alphavbeta5 integrin. Journal Experimental Medicine. 2004; 200:1539-45; Nandrot E F, Finnemann S C. Lack of alphavbeta5 integrin receptor or its ligand MFG-E8: distinct effects on retinal function. Ophthalmic Research. 2008; 40:120-3). We evaluate the treated retinas for evidence of phenotype rescue initially at 1 month and 2 months following AAV-PRPF31 injection in these animals. To evaluate for evidence of prevention of the RPE degeneration, mice are treated at 1 month of age, and the ultrastructure of the RPE is evaluated for phenotypic rescue at 5, 8 and 11 months following AAV-PRPF31 injection (Graziotto JJ , Farkas M H, Bujakowska K, Deramaudt B M, Zhang Q, Nandrot E F, Inglehearn C F, Bhattacharya S S, Pierce E A. Three gene-targeted mouse models of RNA splicing factor RP show late-onset RPE and retinal degeneration. Investigative Ophthalmology & Visual Science. 2011; 52:190-8). Based on data from asymptomatic carriers of PRPF31 mutations, we anticipate that even a modest increase in PRPF31 level in the treated RPE cells will be therapeutic (Rio F T, Wade N M, Ransijn A, Berson E L, Beckmann J S, Rivolta C. Premature termination codons in PRPF31 cause retinitis pigmentosa via haploinsufficiency due to nonsense-mediated mRNA decay. Journal Clinical Investigation. 2008; 118:1519-31; Vithana E N, Abu-Safieh L, Pelosini L, Winchester E, Hornan D, Bird A C, Hunt D M, Bustin S A, Bhattacharya S S. Expression of PRPF31 mRNA in patients with autosomal dominant retinitis pigmentosa: a molecular clue for incomplete penetrance? Investigative Ophthalmology & Visual Science. 2003; 44:4204-9).

### Example 5. AAV-Mediated Gene Augmentation Therapy to Ameliorates the Defective Phagocytosis Phenotype in Cultured RPE Cells

As described above, there is good evidence that mutations in PRPF31 cause disease via haploinsuffiency, and thus that this form of dominant RP is amenable to treatment with gene augmentation therapy (Wang et al., American Journal Medical Genetics A. 2003; 121A:235-9; Xia et al., Molecular Vision. 2004; 10:361-5; Abu-Safieh et al., MolVis. 2006; 12:384-8; Rivolta et al., Human Mutation. 2006; 27:644-53; Sullivan et al., Investigative Ophthalmology & Visual Science. 2006; 47:4579-88; Rio et al., Human Mutation. 2009; 30:1340-7). Consistent with this hypothesis, the level of PRPF31 expression from the wild-type allele correlates with the severity of disease in patients with mutations in PRPF31 (Rio et al., Journal Clinical Investigation. 2008; 118:1519-31; Venturini et al., PLoS genetics. 2012; 8:e1003040; Rose et al., Scientific reports. 2016; 6:19450). To test this hypothesis, we used AAV-mediated gene augmentation therapy to ameliorate the phenotype in cultured RPE cells.

For these studies, we generated an AAV.CASI.PRPF31 viral vector, and showed that this can produce full length PRPF31 protein in cultured cells. The sequence of this vector is as follows:

| (SEQ ID NO: 34) | |
|---|---|
| CTGCGCGCTCGCTCGCTCACTGAGGCCGCCCGGGCAAAGCCCGGGCGTCGGGCGACCTTTGGTCG | |
| CCCGGCCTCAGTGAGCGAGCGAGCGCGCAGAGAGGGAGTGGCCAACTCCATCACTAGGGGTTCC | |
| TTGTAGTTAATGATTAACCCGCCATGCTACTTATCTACGTAGCCATGCTCTAGGAAGATCGGAAT | |
| TCGGAGTTCCGCGTTACATAACTTACGGTAAATGGCCCGCCTGGCTGACCGCCCAACGACCCCCG | |
| CCCATTGACGTCAATAATGACGTATGTTCCCATAGTAACGCCAATAGGGACTTTCCATTGACGTC | |
| AATGGGTGGAGTATTTACGGTAAACTGCCCACTTGGCAGTACATCAAGTGTATCATATGCCAAGT | |
| ACGCCCCCTATTGACGTCAATGACGGTAAATGGCCCGCCTGGCATTATGCCCAGTACATGACCTT | |
| ATGGGACTTTCCTACTTGGCAGTACATCTACGTATTAGTCATCGCTATTACCATGGTCGAGGTGA | |
| GCCCCACGTTCTGCTTCACTCTCCCCATCTCCCCCCCCTCCCCACCCCCAATTTTGTATTTATTTAT | |
| TTTTTAATTATTTTGTGCAGCGATGGGGGCGGGGGGGGGGGGGGGGCGCGCGCCAGGCGGGGCG | |
| GGGCGGGGCGAGGGGCGGGGCGGGGCGAGGCGGAGAGGTGCGGCGGCAGCCAATCAGAGCGGC | |
| GCGCTCCGAAAGTTTCCTTTTATGGCGAGGCGGCGGCGGCGGCGGCCCTATAAAAAGCGAAGCG | |
| CGCGGCGGGCGGGAGTCGCTGCGCGCTGCCTTCGCCCCGTGCCCCGCTCCGCCGCCGCCTCGCGC | |
| CGCCCGCCCCGGCTCTGACTGACCGCGTTACTAAAACAGGTAAGTCCGGCCTCCGCGCCGGGTTT | |
| TGGCGCCTCCCGCGGGCGCCCCCCTCCTCACGGCGAGCGCTGCCACGTCAGACGAAGGGCGCAG | |
| CGAGCGTCCTGATCCTTCCGCCCGGACGCTCAGGACAGCGGCCCGCTGCTCATAAGACTCGGCCT | |
| TAGAACCCCAGTATCAGCAGAAGGACATTTTAGGACGGGACTTGGGTGACTCTAGGGCACTGGT | |
| TTTCTTTCCAGAGAGCGGAACAGGCGAGGAAAAGTAGTCCCTTCTCGGCGATTCTGCGGAGGGAT | |
| CTCCGTGGGGCGGTGAACGCCGATGATGCCTCTACTAACCATGTTCATGTTTTCTTTTTTTTTCTA | |
| CAGGTCCTGGGTGACGAACAGGCTAGCGCCACCATGGGTAAGCCTATCCCTAACCCTCTCCTCGG | |
| TCTCGATTCTACGGCCGCCACCATGTCTCTGGCAGATGAGCTCTTAGCTGATCTCGAAGAGGCAG | |
| CAGAAGAGGAGGAAGGAGGAAGCTATGGGGAGGAAGAAGAGGAGCCAGCGATCGAGGATGTG | |
| CAGGAGGAGACACAGCTGGATCTTTCCGGGGATTCAGTCAAGACCATCGCCAAGCTATGGGATA | |
| GTAAGATGTTTGCTGAGATTATGATGAAGATTGAGGAGTATATCAGCAAGCAAGCCAAAGCTTC | |
| AGAAGTGATGGGACCAGTGGAGGCCGCGCCTGAATACCGCGTCATCGTGGATGCCAACAACCTG | |
| ACCGTGGAGATCGAAAACGAGCTGAACATCATCCATAAGTTCATCCGGGATAAGTACTCAAAGA | |
| GATTCCCTGAACTGGAGTCCTTGGTCCCCAATGCACTGGATTACATCCGCACGGTCAAGGAGCTG | |
| GGCAACAGCCTGGACAAGTGCAAGAACAATGAGAACCTGCAGCAGATCCTCACCAATGCCACCA | |
| TCATGGTCGTCAGCGTCACCGCCTCCACCACCCAGGGGCAGCAGCTGTCGGAGGAGGAGCTGGA | |
| GCGGCTGGAGGAGGCCTGCGACATGGCGCTGGAGCTGAACGCCTCCAAGCACCGCATCTACGAG | |
| TATGTGGAGTCCCGGATGTCCTTCATCGCACCCAACCTGTCCATCATTATCGGGGCATCCACGGC | |
| CGCCAAGATCATGGGTGTGGCCGGCGGCCTGACCAACCTCTCCAAGATGCCCGCCTGCAACATCA | |
| TGCTGCTCGGGGCCCAGCGCAAGACGCTGTCGGGCTTCTCGTCTACCTCAGTGCTGCCCCACACC | |
| GGCTACATCTACCACAGTGACATCGTGCAGTCCCTGCCACCGGATCTGCGGCGGAAAGCGGCCC | |
| GGCTGGTGGCCGCCAAGTGCACACTGGCAGCCCGTGTGGACAGTTTCCACGAGAGCACAGAAGG | |
| GAAGGTGGGCTACGAACTGAAGGATGAGATCGAGCGCAAATTCGACAAGTGGCAGGAGCCGCC | |
| GCCTGTGAAGCAGGTGAAGCCGCTGCCTGCGCCCCTGGATGGACAGCGGAAGAAGCGAGGCGGC | |
| CGCAGGTACCGCAAGATGAAGGAGCGGCTGGGGCTGACGGAGATCCGGAAGCAGGCCAACCGT | |
| ATGAGCTTCGGAGAGATCGAGGAGGACGCCTACCAGGAGGACCTGGGATTCAGCCTGGGCCACC | |
| TGGGCAAGTCGGGCAGTGGGCGTGTGCGGCAGACACAGGTAAACGAGGCCACCAAGGCCAGGA | |
| TCTCCAAGACGCTGCAGCGGACCCTGCAGAAGCAGAGCGTCGTATATGGCGGGAAGTCCACCAT | |
| CCGCGACCGCTCCTCGGGCACGGCCTCCAGCGTGGCCTTCACCCCACTCCAGGGCCTGGAGATTG | |
| TGAACCCACAGGCGGCAGAGAAGAAGGTGGCTGAGGCCAACCAGAAGTATTTCTCCAGCATGGC | |
| TGAGTTCCTCAAGGTCAAGGGCGAGAAGAGTGGCCTTATGTCCACCTGAACCGGTTGGCTAATAA | |
| AGGAAATTTATTTTCATTGCAATAGTGTGTTGGAATTTTTTGTGTCTCTCACTCGGAAGGACATAT | |
| GGGAGGGCAAATCATTTAAAACATCAGAATGAGTATTTGGTTTAGAGTTTGGCAACATATGCCCA | |
| TATGCTGGCTGCCATGAACAAAGGTTGGCTATAAAGAGGTCATCAGTATATGAAACAGCCCCCTG | |
| CTGTCCATTCCTTATTCCATAGAAAAGCCTTGACTTGAGGTTAGATTTTTTTTATATTTTGTTTTGT | |
| GTTATTTTTTTCTTTAACATCCCTAAAATTTTCCTTACATGTTTTACTAGCCAGATTTTTCCTCCTCT | |
| CCTGACTACTCCCAGTCATAGCTGTCCCTCTTCTCTTATGGAGATCGGATCCGAATTCCCGATAAG | |
| GATCTTCCTAGAGCATGGCTACGTAGATAAGTAGCATGGCGGGTTAATCATTAACTACAAGGAAC | |
| CCCTAGTGATGGAGTTGGCCACTCCCTCTCTGCGCGCTCGCTCGCTCACTGAGGCCGGGCGACCA | |
| AAGGTCGCCCGACGCCCGGGCTTTGCCCGGGCGGCCTCAGTGAGCGAGCGAGCGCGCAGCCTTA | |
| ATTAACCTAATTCACTGGCCGTCGTTTTACAACGTCGTGACTGGGAAAACCCTGGCGTTACCCAA | |
| CTTAATCGCCTTGCAGCACATCCCCCTTTCGCCAGCTGGCGTAATAGCGAAGAGGCCCGCACCGA | |
| TCGCCCTTCCCAACAGTTGCGCAGCCTGAATGGCGAATGGGACGCGCCCTGTAGCGGCGCATTAA | |
| GCGCGGCGGGTGTGGTGGTTACGCGCAGCGTGACCGCTACACTTGCCAGCGCCCTAGCGCCCGCT | |
| CCTTTCGCTTTCTTCCCTTCCTTTCTCGCCACGTTCGCCGGCTTTCCCCGTCAAGCTCTAAATCGGG | |
| GGCTCCCTTTAGGGTTCCGATTTAGTGCTTTACGGCACCTCGACCCCAAAAAACTTGATTAGGGT | |
| GATGGTTCACGTAGTGGGCCATCGCCCTGATAGACGGTTTTTCGCCCTTTGACGTTGGAGTCCAC | |
| GTTCTTTAATAGTGGACTCTTGTTCCAAACTGGAACAACACTCAACCCTATCTCGGTCTATTCTTT | |
| TGATTTATAAGGGATTTTGCCGATTTCGGCCTATTGGTTAAAAAATGAGCTGATTTAACAAAAAT | |
| TTAACGCGAATTTTAACAAAATATTAACGTTTATAATTTCAGGTGGCATCTTTCGGGGAAATGTG | |
| CGCGGAACCCCTATTTGTTTATTTTTCTAAATACATTCAAATATGTATCCGCTCATGAGACAATAA | |
| CCCTGATAAATGCTTCAATAATATTGAAAAAGGAAGAGTATGAGTATTCAACATTTCCGTGTCGC | |
| CCTTATTCCCTTTTTTGCGGCATTTTGCCTTCCTGTTTTTGCTCACCCAGAAACGCTGGTGAAAGTA | |
| AAAGATGCTGAAGATCAGTTGGGTGCACGAGTGGGTTACATCGAACTGGATCTCAATAGTGGTA | |
| AGATCCTTGAGAGTTTTCGCCCCGAAGAACGTTTTCCAATGATGAGCACTTTTAAAGTTCTGCTAT | |
| GTGGCGCGGTATTATCCCGTATTGACGCCGGGCAAGAGCAACTCGGTCGCCGCATACACTATTCT | |
| CAGAATGACTTGGTTGAGTACTCACCAGTCACAGAAAAGCATCTTACGGATGGCATGACAGTAA | |
| GAGAATTATGCAGTGCTGCCATAACCATGAGTGATAACACTGCGGCCAACTTACTTCTGACAACG | |
| ATCGGAGGACCGAAGGAGCTAACCGCTTTTTTGCACAACATGGGGGATCATGTAACTCGCCTTGA | |
| TCGTTGGGAACCGGAGCTGAATGAAGCCATACCAAACGACGAGCGTGACACCACGATGCCTGTA | |
| GTAATGGTAACAACGTTGCGCAAACTATTAACTGGCGAACTACTTACTCTAGCTTCCCGGCAACA | |
| ATTAATAGACTGGATGGAGGCGGATAAAGTTGCAGGACCACTTCTGCGCTCGGCCCTTCCGGCTG | |
| GCTGGTTTATTGCTGATAAATCTGGAGCCGGTGAGCGTGGGTCTCGCGGTATCATTGCAGCACTG | |
| GGGCCAGATGGTAAGCCCTCCCGTATCGTAGTTATCTACACGACGGGGAGTCAGGCAACTATGG | |
| ATGAACGAAATAGACAGATCGCTGAGATAGGTGCCTCACTGATTAAGCATTGGTAACTGTCAGA | |
| CCAAGTTTACTCATATATACTTTAGATTGATTTAAAACTTCATTTTTAATTTAAAAGGATCTAGGT | |
| GAAGATCCTTTTTGATAATCTCATGACCAAAATCCCTTAACGTGAGTTTTCGTTCCACTGAGCGTC | |
| AGACCCCGTAGAAAAGATCAAAGGATCTTCTTGAGATCCTTTTTTTCTGCGCGTAATCTGCTGCTT | |
| GCAAACAAAAAAACCACCGCTACCAGCGGTGGTTTGTTTGCCGGATCAAGAGCTACCAACTCTTT | |
| TTCCGAAGGTAACTGGCTTCAGCAGAGCGCAGATACCAAATACTGTCCTTCTAGTGTAGCCGTAG | |
| TTAGGCCACCACTTCAAGAACTCTGTAGCACCGCCTACATACCTCGCTCTGCTAATCCTGTTACCA | |
| GTGGCTGCTGCCAGTGGCGATAAGTCGTGTCTTACCGGGTTGGACTCAAGACGATAGTTACCGGA | |
| TAAGGCGCAGCGGTCGGGCTGAACGGGGGGTTCGTGCACACAGCCCAGCTTGGAGCGAACGACC | |
| TACACCGAACTGAGATACCTACAGCGTGAGCTATGAGAAAGCGCCACGCTTCCCGAAGGGAGAA | |
| AGGCGGACAGGTATCCGGTAAGCGGCAGGGTCGGAACAGGAGAGCGCACGAGGGAGCTTCCAG | |
| GGGGAAACGCCTGGTATCTTTATAGTCCTGTCGGGTTTCGCCACCTCTGACTTGAGCGTCGATTTT | |
| TGTGATGCTCGTCAGGGGGGCGGAGCCTATGGAAAAACGCCAGCAACGCGGCCTTTTTACGGTTC | |
| CTGGCCTTTTGCTGCGGTTTTGCTCACATGTTCTTTCCTGCGTTATCCCCTGATTCTGTGGATAACC | |
| GTATTACCGCCTTTGAGTGAGCTGATACCGCTCGCCGCAGCCGAACGACCGAGCGCAGCGAGTC | |
| AGTGAGCGAGGAAGCGGAAGAGCGCCCAATACGCAAACCGCCTCTCCCCGCGCGTTGGCCGATT | |
| CATTAATGCAGCTGGCACGACAGGTTTCCCGACTGGAAAGCGGGCAGTGAGCGCAACGCAATTA | |
| ATGTGAGTTAGCTCACTCATTAGGCACCCCAGGCTTTACACTTTATGCTTCCGGCTCGTATGTTGT | |
| GTGGAATTGTGAGCGGATAACAATTTCACACAGGAAACAGCTATGACCATGATTACGCCAGATTT | |
| AATTAAGG | |
| ITR-pZac2.1 inverted terminal repeat- | nts 1-130 and 3291-3420 |
| Promoter-CASI | nts 197-1252 |
| Tag-V5 | nts 1259-1309 |
| Insert-PRPF31 | nts 1319-2818 |
| polyA sequence-rabbit β-g1obin | nts 2825-3211 |

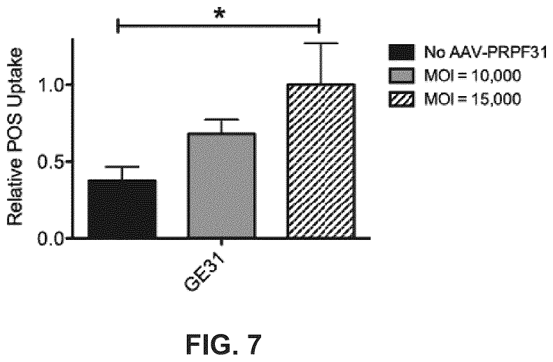

We next tested the ability of the AAV.CASI.PRPF31 to correct the defective phagocytosis phenotype in genome-edited PRFP31-deficient ARPE-19 cells. For these experiments, genome-edited PRPF31 mutant (GE31) ARPE-19 cells were transduced with AAV.CASI.PRPF31 at a multiplicity of infection (MOI) of 0, 10,000, and 15,000. Following transduction, each replicate was incubated with 1×10⁶ FITC-labeled photoreceptor outer segments (FITC-POS) for 1 hour at 37° C. FITC-POS uptake was determined by counting FITC positive cells using flow cytometry. Treatment of the GE31 mutant cell line resulted in increased FITC-POS uptake, in a dose-dependent fashion (FIG. 7). This result confirms the potential of gene augmentation therapy to be used for treating PRPF31-associated retinal degeneration.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of treating retinitis pigmentosa caused by mutations in PRPF31 in a human subject, the method comprising delivering to the eye of the subject a therapeutically effective amount of an Adeno-associated virus type 2 (AAV2) vector comprising a sequence encoding human PRPF31, operably linked to a promoter that drives expression in retinal pigment epithelial (RPE) cells.

2. The method of claim 1 wherein the promoter is a CAG, CASI, RPE65 or VMD2 promotor.

3. The method of claim 2, wherein the PRPF31 sequence is codon optimized.

4. The method of claim 1, wherein the vector is delivered via sub-retinal injection.

5. A method of increasing expression of PRPF31 in the eye of a human subject, the method comprising delivering to the eye of the subject a therapeutically effective amount of an Adeno-associated virus type 2 (AAV2) vector comprising a sequence encoding human PRPF31, operably linked to a promoter that drives expression in retinal pigment epithelial (RPE) cells.

6. The method of claim 5, wherein the promoter is a CAG, CASI, RPE65 or VMD2 promotor.

7. The method of claim 5, wherein the PRPF31 sequence is codon optimized.

8. The method of claim 5, wherein the vector is delivered via sub-retinal injection.

9. An Adeno-associated virus type 2 (AAV2) vector comprising a sequence encoding human PRPF31, operably linked to a promotor that drives expression in retinal pigment epithelial (RPE) cells.

10. The vector of claim 9, wherein the promotor is a CAG, CASI, RPE65 or VMD2 promotor.

11. The vector of claim 9, wherein the PRPF31 sequence is codon optimized.

12. A pharmaceutical composition comprising the vector of claim 9, formulated for delivery via sub-retinal injection.

13. The vector of claim 9, for use in treating retinitis pigmentosa caused by mutations in PRPF31 in the eye of a human subject.

14. The vector of claim 9, for use in increasing expression of PRPF31 in the eye of a human subject.

15. The pharmaceutical composition of claim 12, for use in treating retinitis pigmentosa caused by mutations in PRPF31 in the eye of a human subject.

16. The pharmaceutical composition of claim 12, for use in increasing expression of PRPF31 in the eye of a human subject.


SEQUENCE LISTING (45 sequences)

SEQ ID NO: 1 - protein, 736 aa, Adeno-associated virus
  VARIANT 168: note = /replace="Arg"
  VARIANT 204: note = /replace="Ser"
  VARIANT 266: note = /replace="Gly"
  VARIANT 311: note = /replace="Lys"
  VARIANT 411: note = /replace="Gln"
  VARIANT 460: note = /replace="Glu"
  VARIANT 493: note = /replace="Thr"
  VARIANT 562: note = /replace="Asn"
  VARIANT 576: note = /replace="Glu"
  VARIANT 587: note = /replace="Ala"
  VARIANT 609: note = /replace="Asp"
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKKGQQPAKKRLNFGQTGDSESVPDPQPLGEPPAAPSGVGSNTMAAGGGAPMADNNEGADGVGNASGNWHCDSTWLGDRVITTSTRTWALPTYNNHLYKQISSQSGASTNDNTYFGYSTPWGYFDFNRFHCHFSPRDWQRLINNNWGFRPKRLNFKLFNIQVKEVTTNDGTTTIANNLTSTVQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQTTSGTAGNRTLQFSQAGPSSMANQAKNWLPGPCYRQQRVSKTANQNNNSNFAWTGATKYHLNGRDSLVNPGPAMATHKDDEDKFFPMSGVLIFGKQGAGNSNVDLDNVMITSEEEIKTTNPVATEQYGTVATNLQSSNTAPATGTVNSQGALPGMVWQNRDVYLQGPIWAKIPHTDGHFHPSPLMGGFGLKHPPPQILIKNTPVPANPPTTFSPAKFASFITQYSTGQVSVEIEWELQKENSKRWNPEIQYTSNYNKSTNVDFAVDTNGVYSEPRPIGTRYLTRNL

SEQ ID NO: 2 - DNA, 2208 bp, Adeno-associated virus
  variation 502..504: note = /replace="aaa"
  variation 610..612: note = /replace="agc"
  variation 796..798: note = /replace="ggc"
  variation 931..933: note = /replace="aag"
  variation 1231..1233: note = /replace="cag"
  variation 1378..1380: note = /replace="gag"
  variation 1477..1479: note = /replace="acc"
  variation 1684..1686: note = /replace="aac"
  variation 1726..1728: note = /replace="gag"
  variation 1759..1761: note = /replace="gcc"
  variation 1825..1827: note = /replace="gac"
  source 1..2208: mol_type = other DNA
atggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagcaatcaccccaggaaccagactcctcttcgggcatcggcaagaaaggccagcagcccgcgaagaagagactcaactttgggcagacaggcgactcagagtcagtgcccgaccctcaaccactcggagaaccccccgcagccccctctggtgtgggatctaatacaatggcagcaggcggtggcgctccaatggcagacaataacgaaggcgccgacggagtgggtaacgcctcaggaaattggcattgcgattccacatggctgggcgacagagtcatcaccaccagcacccgaacctgggccctccccacctacaacaaccacctctacaagcaaatctccagccaatcgggagcaagcaccaacgacaacacctacttcggctacagcaccccctgggggtattttgactttaacagattccactgccacttctcaccacgtgactggcagcgactcatcaacaacaactggggattccggcccaagagactcaacttcaagctcttcaacatccaggtcaaggaggtcacgacgaatgatggcaccacgaccatcgccaataaccttaccagcacggttcaggtctttacggactcggaataccagctcccgtacgtcctcggctctgcgcaccagggctgcctgcctccgttcccggcggacgtcttcatgattcctcagtacgggtacctgactctgaacaatggcagtcaggccgtgggccgttcctccttctactgcctggagtactttccttctcaaatgctgagaacgggcaacaactttgagttcagctacacgtttgaggacgtgccttttcacagcagctacgcgcacagccaaagcctggaccggctgatgaaccccctcatcgaccagtacctgtactacctgtctcggactcagaccacgagtggtaccgcaggaaatcggacgttgcaattttctcaggccgggcctagtagcatggcgaatcaggccaaaaactggctacccgggccctgctaccggcagcaacgcgtctccaagacagcgaatcaaaataacaacagcaactttgcctggaccggtgccaccaagtatcatctgaatggcagagactctctggtaaatcccggtcccgctatggcaacccacaaggacgacgaagacaaattttttccgatgagcggagtcttaatatttgggaaacagggagctggaaatagcaacgtggaccttgacaacgttatgataaccagtgaggaagaaattaaaaccaccaacccagtggccacagaacagtacggcacggtggccactaacctgcaatcgtcaaacaccgctcctgctacagggaccgtcaacagtcaaggagccttacctggcatggtctggcagaaccgggacgtgtacctgcagggtcctatctgggccaagattcctcacacggacggacactttcatccctcgccgctgatgggaggctttggactgaaacacccgcctcctcagatcctgattaagaatacacctgttcccgcgaatcctccaactaccttcagtccagctaagtttgcgtcgttcatcacgcagtacagcaccggacaggtcagcgtggaaattgaatgggagctgcagaaagaaaacagcaaacgctggaacccagagattcaatacacttccaactacaacaaatctacaaatgtggactttgctgttgacacaaatggcgtttattctgagcctcgccccatcggcacccgttacctcacccgtaatctg

SEQ ID NO: 3 - protein, 737 aa, Adeno-associated virus
  VARIANT 157: note = /replace="Ser"
  VARIANT 168: note = /replace="Arg"
  VARIANT 262: note = /replace="Ser"
  VARIANT 263: note = /replace="His"
  VARIANT 312: note = /replace="Lys"
  VARIANT 412: note = /replace="Gln"
  VARIANT 460: note = /replace="Gln"
  VARIANT 461: note = /replace="Glu"
  VARIANT 552: note = /replace="Ser"
  VARIANT 556: note = /replace="Tyr"
  VARIANT 557: note = /replace="Ser"
  VARIANT 563: note = /replace="Asn"
  VARIANT 580: note = /replace="Ile"
  VARIANT 588: note = /replace="Ser"
  VARIANT 664: note = /replace="Thr"
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSTGIGKKGQQPAKKRLNFGQTGDSESVPDPQPLGEPPAAPSGVGSNTMAAGGGAPMADNNEGADGVGNASGNWHCDSTWLGDRVITTSTRTWALPTYNNHLYKQISNSQSGGSTNDNTYFGYSTPWGYFDFNRFHCHFSPRDWQRLINNNWGFRPKRLNFKLFNIQVKEVTTNDGTTTIANNLTSTVQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQTTGGTAGNRTLQFSQAGPSSMANQAKNWLPGPCYRQQRVSKTTNQNNNSNFAWTGATKYHLNGRDSLVNPGVAMATHKDDEDRFFPSSGVLIFGKQGAGNDNVDLDNVMITSEEEIKTTNPVATEEYGVVATNLQSANTAPQTGTVNSQGALPGMVWQNRDVYLQGPIWAKIPHTDGNFHPSPLMGGFGLKHPPPQILIKNTPVPANPPTTFSPAKFASFITQYSTGQVSVEIEWELQKENSKRWNPEIQYTSNYNKSTNVDFAVDTEGVYSEPRPIGTRYLTRNL

SEQ ID NO: 4 - DNA, 2211 bp, Adeno-associated virus
  variation 469..471: note = /replace="agc"
  variation 502..504: note = /replace="aag"
  variation 784..786: note = /replace="agt"
  variation 787..789: note = /replace="cac"
  variation 934..936: note = /replace="aag"
  variation 1234..1236: note = /replace="cag"
  variation 1378..1380: note = /replace="cag"
  variation 1381..1383: note = /replace="gag"
  variation 1654..1656: note = /replace="agc"
  variation 1666..1668: note = /replace="tac"
  variation 1669..1671: note = /replace="agc"
  variation 1687..1689: note = /replace="aac"
  variation 1738..1740: note = /replace="atc"
  variation 1762..1764: note = /replace="agc"
  variation 1990..1992: note = /replace="acc"
  source 1..2211: mol_type = other DNA
atggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagcaatcaccccaggaaccagactcctctacgggcatcggcaagaaaggccagcagcccgcgaaaaagagactcaactttgggcagactggcgactcagagtcagtgcccgaccctcaaccactcggagaaccccccgcagccccctctggtgtgggatctaatacaatggctgcaggcggtggcgctccaatggcagacaataacgaaggcgccgacggagtgggtaatgcctcaggaaattggcattgcgattccacatggctgggcgacagagtcatcaccaccagcacccgaacctgggccctccccacctacaacaaccacctctacaagcaaatctccaacagccaatcgggaggaagcaccaacgacaacacctacttcggctacagcaccccctgggggtattttgactttaacagattccactgccacttctcaccacgtgactggcagcgactcatcaacaacaactggggattccggcccaagagactcaacttcaagctcttcaacatccaggtcaaggaggtcacgacgaatgatggcaccacgaccatcgccaataaccttaccagcacggttcaggtctttacggactcggaataccagctcccgtacgtcctcggctctgcgcaccagggctgcctgcctccgttcccggcggacgtcttcatgattcctcagtacgggtacctgactctgaacaatggcagtcaggccgtgggccgttcctccttctactgcctggagtactttccttctcaaatgctgagaacgggcaacaactttgagttcagctacacgtttgaggacgtgccttttcacagcagctacgcgcacagccaaagcctggaccggctgatgaaccccctcatcgaccagtacctgtactacctgtctcggactcagaccacgggaggtaccgcaggaaatcggacgttgcaattttctcaggccgggcctagtagcatggcgaatcaggccaaaaactggctacccgggccctgctaccggcagcaacgcgtctccaagacaacgaatcaaaataacaacagcaactttgcctggaccggtgccaccaagtatcatctgaatggcagagactctctggtaaatcccggtgtcgctatggcaacccacaaggacgacgaagaccgattttttccgtccagcggagtcttaatatttgggaaacagggagctggaaatgacaacgtggaccttgacaacgttatgataaccagtgaggaagaaattaaaaccaccaacccagtggccacagaagagtacggcgtggtggccactaacctgcaatcggcaaacaccgctcctcaaacagggaccgtcaacagtcaaggagccttacctggcatggtctggcagaaccgggacgtgtacctgcagggtcctatctgggccaagattcctcacacggacggaaactttcatccctcgccgctgatgggaggctttggactgaaacacccgcctcctcagatcctgattaagaatacacctgttcccgcgaatcctccaactaccttcagtccagctaagtttgcgtcgttcatcacgcagtacagcaccggacaggtcagcgtggaaattgaatgggagctgcagaaagaaaacagcaaacgctggaacccagagattcaatacacttccaactacaacaaatctacaaatgtggactttgctgttgacacagaaggcgtttattctgagcctcgccccatcggcacccgttacctcacccgtaatctg

SEQ ID NO: 5 - protein, 738 aa, Adeno-associated virus
  VARIANT 158: note = /replace="Ser"
  VARIANT 169: note = /replace="Arg"
  VARIANT 564: note = /replace="Asn"
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQREPDSSTGIGKKGQQPAKKRLNFGQTGDSESVPDPQPLGEPPAAPSGVGSNTMAAGGGAPMADNNEGADGVGNSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTTNEGTKTIANNLTSTVQVFTDSEYQLPYVLGSAHQGCLPPFPADVFMIPQYGYLTLNNGSQAVGRSSFYCLEYFPSQMLRTGNNFQFSYTFEDVPFHSSYAHSQSLDRLMNPLIDQYLYYLSRTQTTGGTAGTQTLQFSQAGPSSMANQAKNWLPGPCYRQQRVSTTTNQNNNSNFAWTGATKYHLNGRDSLVNPGVAMATHKDDEDRFFPSSGVLIFGKQGAGNDNVDYSNVMITSEEEIKTTNPVATEEYGVVATNLQSANTAPQTGTVNSQGALPGMVWQNRDVYLQGPIWAKIPHTDGNFHPSPLMGGFGLKHPPPQILIKNTPVPADPPTTFNQAKLNSFITQYSTGQVSVEIEWELQKENSKRWNPEIQYTSNYYKSTNVDFAVNTEGVYSEPRPIGTRYLTRNL

SEQ ID NO: 6 - DNA, 2214 bp, Adeno-associated virus
  variation 472..474: note = /replace="agc"
  variation 505..507: note = /replace="aga"
  variation 1690..1692: note = /replace="aac"
  source 1..2214: mol_type = other DNA
atggctgccgatggttatcttccagattggctcgaggacaacctctctgagggcattcgcgagtggtgggacctgaaacctggagccccgaaacccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataatcacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagcagtcaccacagcgtgagcccgactcctccacgggcatcggcaagaaaggccagcagcccgccaaaaagagactcaatttcggtcagactggcgactcagagtcagtccccgaccctcaacctctcggagaacctccagcagcgccctctggtgtgggatctaatacaatggctgcaggcggtggcgcaccaatggcagacaataacgaaggtgccgacggagtgggtaattcctcgggaaattggcattgcgattccacatggctgggcgacagagtcatcaccaccagcacccgaacctgggccctgcccacctacaacaaccacctctacaagcaaatctccaacgggacctcgggaggcagcaccaacgacaacacctactttggctacagcaccccctgggggtattttgactttaacagattccactgccacttctcaccacgtgactggcagcgactcatcaacaacaactggggattccggcccaagagactcaacttcaagctcttcaacatccaggtcaaagaggtcacgacgaatgaaggcaccaagaccatcgccaataacctcaccagcaccgtccaggtgtttacggactcggaataccagctgccgtacgtcctcggctctgcccaccagggctgcctgcctccgttcccggcggacgtcttcatgattcctcagtacggctacctgactctcaacaacggtagtcaggccgtgggacgttcctccttctactgcctggagtacttcccctctcagatgctgagaacgggcaacaactttcaattcagctacactttcgaggacgtgcctttccacagcagctacgcgcacagccagagtttggacaggctgatgaatcctctcatcgaccagtacctgtactacctgtcaagaacccagactacgggaggcacagcgggaacccagacgttgcagttttctcaggccgggcctagcagcatggcgaatcaggccaaaaactggctgcctggaccctgctacagacagcagcgcgtctccacgacaacgaatcaaaacaacaacagcaactttgcctggactggtgccaccaagtatcatctgaacggcagagactctctggtgaatccgggcgtcgccatggcaacccacaaggacgacgaggaccgcttcttcccatccagcggcgtcctcatatttggcaagcagggagctggaaatgacaacgtggactatagcaacgtgatgataaccagcgaggaagaaatcaagaccaccaaccccgtggccacagaagagtatggcgtggtggctactaacctacagtcggcaaacaccgctcctcaaacggggaccgtcaacagccagggagccttacctggcatggtctggcagaaccgggacgtgtacctgcagggtcctatttgggccaagattcctcacacagatggcaactttcacccgtctcctttaatgggcggctttggacttaaacatccgcctcctcagatcctcatcaaaaacactcctgttcctgcggatcctccaacaacgttcaaccaggccaagctgaattctttcatcacgcagtacagcaccggacaagtcagcgtggagatcgagtgggagctgcagaaggagaacagcaagcgctggaacccagagattcagtatacttccaactactacaaatctacaaatgtggactttgctgttaatactgagggtgtttactctgagcctcgccccattggcactcgttacctcacccgtaatctg

SEQ ID NO: 7 - protein, 738 aa, Adeno-associated virus
  VARIANT 158: note = /replace="Ser"
  VARIANT 169: note = /replace="Lys"
  VARIANT 315: note = /replace="Ser"
  VARIANT 413: note = /replace="Glu"
  VARIANT 472: note = /replace="Thr" or "Ser"
  VARIANT 534: note = /replace="Glu"
  VARIANT 542: note = /replace="Val"
  VARIANT 595: note = /replace="Val"
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQREPDSSTGIGKKGQQPARKRLNFGQTGDSESVPDPQPLGEPPAAPSGVGSNTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFSYTFEDVPFHSSYAHSQSLDRLMNPLIDQYLYYLSRTQTTGGTAGTQTLQFSQAGPSNMANQAKNWLPGPCYRQQRVSTTTSQNNNSNFAWTGATKYHLNGRDSLVNPGVAMATHKDDEDRFFPSSGILIFGKQGAGKDNVDYSNVMLTSEEEIKTTNPVATEEYGVVADNLQQQNTAPQIGTVNSQGALPGMVWQNRDVYLQGPIWAKIPHTDGNFHPSPLMGGFGLKHPPPQILIKNTPVPADPPTTFNQAKLNSFITQYSTGQVSVEIEWELQKENSKRWNPEIQYTSNYYKSTNVDFAVNTEGVYSEPRPIGTRYLTRNL

SEQ ID NO: 8 - DNA, 2214 bp, Adeno-associated virus
  variation 472..474: note = /replace="agc"
  variation 505..507: note = /replace="aag"
  variation 943..945: note = /replace="agc"
  variation 1237..1239: note = /replace="gaa"
  variation 1414..1416: note = /replace="aac" or "agc"
  variation 1600..1602: note = /replace="gag"
  variation 1624..1626: note = /replace="gtc"
  variation 1783..1785: note = /replace="gta"
  source 1..2214: mol_type = other DNA
atggctgccgatggttatcttccagattggctcgaggacaacctctctgagggcattcgcgagtggtgggacctgaaacctggagccccgaaacccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataatcacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagcagtcaccacagcgtgagcccgactcctccacgggcatcggcaagaaaggccagcagcccgccagaaagagactcaatttcggtcagactggcgactcagagtcagtccccgaccctcaacctctcggagaacctccagcagcgccctctggtgtgggatctaatacaatggctgcaggcggtggcgcaccaatggcagacaataacgaaggtgccgacggagtgggtagttcctcgggaaattggcattgcgattccacatggctgggcgacagagtcatcaccaccagcacccgaacctgggccctgcccacctacaacaaccacctctacaagcaaatctccaacgggacctcgggaggcagcaccaacgacaacacctactttggctacagcaccccctgggggtattttgactttaacagattccactgccacttctcaccacgtgactggcagcgactcatcaacaacaactggggattccggcccaagagactcaacttcaagctcttcaacatccaggtcaaagaggtcacgcagaatgaaggcaccaagaccatcgccaataacctcaccagcaccatccaggtgtttacggactcggaataccagctgccgtacgtcctcggctctgcccaccagggctgcctgcctccgttcccggcggacgtcttcatgattcctcagtacggctacctgactctcaacaacggtagtcaggccgtgggacgttcctccttctactgcctggagtacttcccctctcagatgctgagaacgggcaacaactttcaattcagctacactttcgaggacgtgcctttccacagcagctacgcgcacagccagagtttggacaggctgatgaatcctctcatcgaccagtacctgtactacctgtcaagaacccagactacgggaggcacagcgggaacccagacgttgcagttttctcaggccgggcctagcaacatggcgaatcaggccaaaaactggctgcctggaccctgctacagacagcagcgcgtctccacgacaacgtcgcaaaacaacaacagcaactttgcctggactggtgccaccaagtatcatctgaacggcagagactctctggtgaatccgggcgtcgccatggcaacccacaaggacgacgaggaccgcttcttcccatccagcggcatcctcatatttggcaagcagggagctggaaaagacaacgtggactatagcaacgtgatgctaaccagcgaggaagaaatcaagaccaccaaccccgtggccacagaagagtatggcgtggtggctgataacctacagcagcaaaacaccgctcctcaaatagggaccgtcaacagccagggagccttacctggcatggtctggcagaaccgggacgtgtacctgcagggtcctatttgggccaagattcctcacacagatggcaactttcacccgtctcctttaatgggcggctttggacttaaacatccgcctcctcagatcctcatcaaaaacactcctgttcctgcggatcctccaacaacgttcaaccaggccaagctgaattctttcatcacgcagtacagcaccggacaagtcagcgtggagatcgagtgggagctgcagaaggagaacagcaagcgctggaacccagagattcagtatacttccaactactacaaatctacaaatgtggactttgctgttaatactgagggtgtttactctgagcctcgccccattggcactcgttacctcacccgtaatctg

SEQ ID NO: 9 - protein, 738 aa, Adeno-associated virus
  VARIANT 169: note = /replace="Lys"
  VARIANT 315: note = /replace="Ser"
  VARIANT 534: note = /replace="Glu"
  VARIANT 542: note = /replace="Val"
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPIGEPPAAPSGVGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTAGTQQLLFSQAGPSNMSAQAKNWLPGPCYRQQRVSTTLSQNNNSNFAWTGATKYHLNGRDSLVNPGVAMATHKDDEDRFFPSSGILMFGKQGAGKDNVDYSNVMLTSEEEIKTTNPVATEQYGVVADNLQQQNTAPIVGAVNSQGALPGMVWQNRDVYLQGPIWAKIPHTDGNFHPSPLMGGFGLKHPPPQILIKNTPVPADPPTTFNQAKLNSFITQYSTGQVSVEIEWELQKENSKRWNPEIQYTSNYYKSTNVDFAVNTEGVYSEPRPIGTRYLTRNL

SEQ ID NO: 10 - DNA, 2214 bp, Adeno-associated virus
  variation 505..507: note = /replace="aaa"
  variation 943..945: note = /replace="agc"
  variation 1600..1602: note = /replace="gag"
  variation 1624..1626: note = /replace="gtc"
  source 1..2214: mol_type = other DNA
atggctgccgatggttatcttccagattggctcgaggacaacctctctgagggcattcgcgagtggtgggacctgaaacctggagccccgaaacccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataatcacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccgtcaccacagcgttcccccgactcctccacgggcatcggcaagaaaggccagcagcccgccagaaagagactcaatttcggtcagactggcgactcagagtcagtccccgaccctcaacctatcggagaacctccagcagcgccctctggtgtgggatctggtacaatggctgcaggcggtggcgcaccaatggcagacaataacgaaggtgccgacggagtgggtagttcctcgggaaattggcattgcgattccacatggctgggcgacagagtcatcaccaccagcacccgaacctgggccctgcccacctacaacaaccacctctacaagcaaatctccaacgggacctcgggaggcagcaccaacgacaacacctactttggctacagcaccccctgggggtattttgactttaacagattccactgccacttctcaccacgtgactggcagcgactcatcaacaacaactggggattccggcccaagagactcaacttcaagctcttcaacatccaggtcaaagaggtcacgcagaatgaaggcaccaagaccatcgccaataacctcaccagcaccatccaggtgtttacggactcggaataccagctgccgtacgtcctcggctctgcccaccagggctgcctgcctccgttcccggcggacgtcttcatgattcctcagtacggctacctgactctcaacaacggtagtcaggccgtgggacgttcctccttctactgcctggagtacttcccctctcagatgctgagaacgggcaacaactttgagttcagctacactttcgaggacgtgcctttccacagcagctacgcgcacagccagagtttggacaggctgatgaatcctctcatcgaccagtacctgtactacctgtcaagaacccagtctacgggaggcacagcgggaacccagcagttgctgttttctcaggccgggcctagcaacatgtcggctcaggccaaaaactggctgcctggaccctgctacagacagcagcgcgtctccacgacactgtcgcaaaacaacaacagcaactttgcctggactggtgccaccaagtatcatctgaacggcagagactctctggtgaatccgggcgtcgccatggcaacccacaaggacgacgaggaccgcttcttcccatccagcggcatcctcatgtttggcaagcagggagctggaaaagacaacgtggactatagcaacgtgatgctaaccagcgaggaagaaatcaagaccaccaaccccgtggccacagaacagtatggcgtggtggctgataacctacagcagcaaaacaccgctcctattgtgggggccgtcaacagccagggagccttacctggcatggtctggcagaaccgggacgtgtacctgcagggtcctatttgggccaagattcctcacacagatggcaactttcacccgtctcctttaatgggcggctttggacttaaacatccgcctcctcagatcctcatcaaaaacactcctgttcctgcggatcctccaacaacgttcaaccaggccaagctgaattctttcatcacgcagtacagcaccggacaagtcagcgtggagatcgagtgggagctgcagaaggagaacagcaagcgctggaacccagagattcagtatacttccaactactacaaatctacaaatgtggactttgctgttaatactgagggtgtttactctgagcctcgccccattggcactcgttacctcacccgtaatctg

SEQ ID NO: 11 - protein, 738 aa, Adeno-associated virus
  VARIANT 471: note = /replace="Asn"
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTAGTQQLLFSQAGPSNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNTAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 12 - DNA, 2214 bp, Adeno-associated virus
  variation 1411..1413: note = /replace="aat"
  source 1..2214: mol_type = other DNA
atggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccagcagcccgcgaaaaagagactcaactttgggcagactggcgactcagagtcagtgcccgaccctcaaccaatcggagaaccccccgcaggcccctctggtctgggatctggtacaatggctgcaggcggtggcgctccaatggcagacaataacgaaggcgccgacggagtgggtagttcctcaggaaattggcattgcgattccacatggctgggcgacagagtcatcaccaccagcacccgaacctgggccctccccacctacaacaaccacctctacaagcaaatctccaacgggacttcgggaggaagcaccaacgacaacacctacttcggctacagcaccccctgggggtattttgactttaacagattccactgccacttctcaccacgtgactggcagcgactcatcaacaacaactggggattccggcccaagagactcaacttcaagctcttcaacatccaggtcaaggaggtcacgcagaatgaaggcaccaagaccatcgccaataaccttaccagcacgattcaggtctttacggactcggaataccagctcccgtacgtcctcggctctgcgcaccagggctgcctgcctccgttcccggcggacgtcttcatgattcctcagtacgggtacctgactctgaacaatggcagtcaggccgtgggccgttcctccttctactgcctggagtactttccttctcaaatgctgagaacgggcaacaactttgagttcagctacacgtttgaggacgtgccttttcacagcagctacgcgcacagccaaagcctggaccggctgatgaaccccctcatcgaccagtacctgtactacctgtctcggactcagtccacgggaggtaccgcaggaactcagcagttgctattttctcaggccgggcctagtaacatgtcggctcaggccaaaaactggctacccgggccctgctaccggcagcaacgcgtctccacgacactgtcgcaaaataacaacagcaactttgcctggaccggtgccaccaagtatcatctgaatggcagagactctctggtaaatcccggtgtcgctatggcaacccacaaggacgacgaagagcgattttttccgtccagcggagtcttaatgtttgggaaacagggagctggaaaagacaacgtggactatagcagcgttatgctaaccagtgaggaagaaattaaaaccaccaacccagtggccacagaacagtacggcgtggtggccgataacctgcaacagcaaaacaccgctcctattgtaggggccgtcaacagtcaaggagccttacctggcatggtctggcagaaccgggacgtgtacctgcagggtcctatctgggccaagattcctcacacggacggaaactttcatccctcgccgctgatgggaggctttggactgaaacacccgcctcctcagatcctgattaagaatacacctgttcccgcggatcctccaactaccttcagtcaagctaagctggcgtcgttcatcacgcagtacagcaccggacaggtcagcgtggaaattgaatgggagctgcagaaagaaaacagcaaacgctggaacccagagattcaatacacttccaactactacaaatctacaaatgtggactttgctgttaacacagaaggcacttattctgagcctcgccccatcggcacccgttacctcacccgtaatctg

SEQ ID NO: 13 - protein, 737 aa, Adeno-associated virus
  VARIANT 148: note = /replace="Gln"
  VARIANT 169: note = /replace="Arg"
  VARIANT 314: note = /replace="Asn"
  VARIANT 466: note = /replace="His"
  VARIANT 563: note = /replace="Ser"
  VARIANT 580: note = /replace="Ile"
  VARIANT 588: note = /replace="Ser"
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPLGEPPAAPSGVGSGTMAAGGGAPMADNNEGADGVGNASGNWHCDSTWLGDRVITTSTRTWALPTYNNHLYKQISSQSAGSTNDNTYFGYSTPWGYFDFNRFHCHFSPRDWQRLINNNWGFRPKKLRFKLFNIQVKEVTTNDGVTTIANNLTSTVQVFSDSEYQLPYVLGSAHQGCLPPFPADVFMIPQYGYLTLNNGSQSVGRSSFYCLEYFPSQMLRTGNNFEFSYTFEDVPFHSSYAHSQSLDRLMNPLIDQYLYYLARTQSTTGGTAGNRELQFYQAGPSTMAEQAKNWLPGPCYRQQRVSKTLDQNNNSNFAWTGATKYHLNGRNSLVNPGVAMATHKDDEDRFFPSSGVLIFGKTGAANKTTLENVLMTNEEEIKTTNPVATEEYGVVSSNLQSANTAPQTQTVNSQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNYDKSTNVDFAVDSEGVYSEPRPIGTRYLTRNL

SEQ ID NO: 14 - DNA, 2211 bp, Adeno-associated virus
  variation 442..444: note = /replace="cag"
  variation 505..507: note = /replace="aga"
  variation 940..942: note = /replace="aac"
  variation 1396..1398: note = /replace="cac"
  variation 1687..1689: note = /replace="agt"
  variation 1738..1740: note = /replace="ata"
  variation 1762..1764: note = /replace="tct"
  source 1..2211: mol_type = genomic DNA
atggctgccgatggttatcttccagattggctcgaggacaacctctctgagggcattcgcgagtggtgggacctgaaacctggagccccgaaacccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcatttgggggcaacctcgggcgagcagtcttccaggccaagaagcgggttctcgaacctctcggtctggttgaggaaggcgctaagacggctcctggaaagaagagaccggtagagccgtcacctcagcgttcccccgactcctccacgggcatcggcaagaaaggccagcagcccgccaaaaagagactcaatttcggtcagactggcgactcagagtcagtccccgaccctcaacctctcggagaacctccagcagcgccctctggtgtgggatctggtacaatggctgcaggcggtggcgcaccaatggcagacaataacgaaggtgccgacggagtgggtaatgcctcaggaaattggcattgcgattccacatggctgggcgacagagtcattaccaccagcacccgaacctgggccctgcccacctacaacaaccacctctacaagcaaatctccagtcaaagtgcaggtagtaccaacgacaacacctacttcggctacagcaccccctgggggtattttgactttaacagattccactgccacttctcaccacgtgactggcagcgactcatcaacaacaactggggattccggcccaagaagctgcggttcaagctcttcaacatccaggtcaaggaggtcacgacgaatgacggcgttacgaccatcgctaataaccttaccagcacggttcaggtattctcggactcggaataccagctgccgtacgtcctcggctctgcgcaccagggctgcctgcctccgttcccggcggacgtcttcatgattcctcagtacggctacctgactctcaacaatggcagtcagtctgtgggacgttcctccttctactgcctggagtacttcccctctcagatgctgagaacgggcaacaactttgagttcagctacaccttcgaggacgtgcctttccacagcagctacgcacacagccagagcctggaccggctgatgaatcccctcatcgaccagtacttgtactacctggccagaacacagagtaccacaggaggcacagctggcaatcgggaactgcagttttaccaggccgggccttcaactatggccgaacaagccaagaattggttacctggaccttgctaccggcaacaaagagtctccaaaacgctggatcaaaacaacaacagcaactttgcttggactggtgccaccaaatatcacctgaacggcagaaactcgttggttaatcccggcgtcgccatggcaactcacaaggacgacgaggaccgctttttcccatccagcggagtcctgatttttggaaaaactggagcagctaacaaaactacattggaaaatgtgttaatgacaaatgaagaagaaattaaaactactaatcctgtagccacggaagaatacggggtagtcagcagcaacttacaatcggctaatactgcaccccagacacaaactgtcaacagccagggagccttacctggcatggtctggcagaaccgggacgtgtacctgcagggtcccatctgggccaagattcctcacacggatggcaactttcacccgtctcctttgatgggcggctttggacttaaacatccgcctcctcagatcctgatcaagaacactcccgttcccgctaatcctccggaggtgtttactcctgccaagtttgcttcgttcatcacacagtacagcaccggacaagtcagcgtggaaatcgagtgggagctgcagaaggaaaacagcaagcgctggaacccggagattcagtacacctccaactatgataagtcgactaatgtggactttgccgttgacagcgagggtgtttactctgagcctcgccctattggcactcgttacctcacccgtaatctg

SEQ ID NO: 15 - protein, 735 aa, Adeno-associated virus
  VARIANT 162: note = /replace="Thr"
  VARIANT 168: note = /replace="Arg"
  VARIANT 224: note = /replace="Ser"
  VARIANT 310: note = /replace="Lys"
  VARIANT 410: note = /replace="Gln"
  VARIANT 446: note = /replace="Asn"
  VARIANT 461: note = /replace="Leu"
  VARIANT 471: note = /replace="Ser"
  VARIANT 708: note = /replace="Thr"
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKSGQQPAKKRLNFGQTGDSESVPDPQPLGEPPAAPSGVGSNTMASGGGAPMADNNEGADGVGNASGNWHCDSTWLGDRVITTSTRTWALPTYNNHLYKQISSQSGASNDNHYFGYSTPWGYFDFNRFHCHFSPRDWQRLINNNWGFRPKRLNFKLFNIQVKEVTTNDGTTTIANNLTSTVQVFTDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLSRTQTTSGTAQNRELQFSQAGPSSMANQAKNWLPGPCYRQQRVSKTANDNNNSNFAWTGATKYHLNGRDSLVNPGPAMASHKDDEDKFFPMSGVLIFGKQGAGASNVDLDNVMITDEEEIKTTNPVATEQYGTVATNLQSSNTAPATGTVNSQGALPGMVWQDRDVYLQGPIWAKIPHTDGHFHPSPLMGGFGLKHPPPQILIKNTPVPANPPTTFSPAKFASFITQYSTGQVSVEIEWELQKENSKRWNPEIQYTSNYNKSANVDFTVDTNGVYSEPRPIGTRYLTRNL

SEQ ID NO: 16 - DNA, 2205 bp, Adeno-associated virus
  variation 484..486: note = /replace="aca"
  variation 502..504: note = /replace="aga"
  variation 670..672: note = /replace="tcc"
  variation 928..930: note = /replace="aaa"
  variation 1228..1230: note = /replace="cag"
  variation 1336..1338: note = /replace="aac"
  variation 1381..1383: note = /replace="ctg"
  variation 1411..1413: note = /replace="tct"
  variation 2122..2124: note = /replace="acc"
  source 1..2205: mol_type = other DNA
atggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggatgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagagggttctcgaacctcttggtctggttgaggaaggtgctaagacggctcctggaaagaaacgtccggtagagcagtcgccacaagagccagactcctcctcgggcattggcaagtcaggccagcagcccgctaaaaagagactcaattttggtcagactggcgactcagagtcagtccccgacccacaacctctcggagaacctccagcagccccctctggtgtgggatctaatacaatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtcaatcaggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcaccacgaccatcgctaataaccttaccagcacggttcaagtcttcacggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaataactttaccttcagctacaccttcgaggacgtgcctttccacagcagctacgcgcacagccagagcctggaccggctgatgaatcctctcatcgaccagtacctgtattacctgagcagaactcagactacgtccggaactgcccaaaacagggagttgcagtttagccaggcgggtccatctagcatggctaatcaggccaaaaactggctacctggaccctgttaccggcagcagcgcgtttctaaaacagcaaatgacaacaacaacagcaactttgcctggactggtgctacaaaatatcaccttaatgggcgtgattctttagtcaaccctggccctgctatggcctcacacaaagacgacgaagacaagttctttcccatgagcggtgtcttgatttttggaaagcagggcgccggagcttcaaacgttgatttggacaatgtcatgatcacagacgaagaggaaatcaaaaccactaaccccgtggccaccgaacaatatgggactgtggcaaccaatctccagagcagcaacacagcccctgcgaccggaactgtgaattctcagggagccttacctggaatggtgtggcaagacagagacgtatacctgcagggtcctatttgggccaaaattcctcacacggatggacactttcacccgtctcctctcatgggcggctttggacttaagcacccgcctcctcagatcctcatcaaaaacacgcctgttcctgcgaatcctccgacaacgttttcgcctgcaaagtttgcttcattcatcacccagtattccacaggacaagtgagcgtggagattgaatgggagctgcagaaagaaaacagcaaacgctggaatcccgaaatacagtatacatctaactataataaatctgccaacgttgatttcactgtggacaccaatggagtttatagtgagcctcgccccattggcacccgttacctcacccgtaacctg

SEQ ID NO: 17 - protein, 735 aa, Adeno-associated virus
  VARIANT 42: note = /replace="Ser"
  VARIANT 168: note = /replace="Lys"
  VARIANT 310: note = /replace="Arg"
  VARIANT 410: note = /replace="Gln"
  VARIANT 446: note = /replace="Arg"
  VARIANT 461: note = /replace="Leu"
  VARIANT 471: note = /replace="Ser"
  VARIANT 475: note = /replace="Arg"
  VARIANT 504: note = /replace="Ala"
  VARIANT 539: note = /replace="Asn"
MAADGYLPDWLEDNLSEGIREWWDLKPGAPQPKANQQHQDDGRGLVLPGYKYLGPFNGLDKGEPVNEADAAALEHDKAYDQQLKAGDNPYLKYNHADAEFQERLQEDTSFGGNLGRAVFQAKKRVLEPLGLVEEAAKTAPGKKRPVEQSPQEPDSSSGIGKSGQQPARKRLNFGQTGDSESVPDPQPLGEPPAAPSGVGSNTMASGGGAPMADNNEGADGVGNSSGNWHCDSTWLGDRVITTSTRTWALPTYNNHLYKQISSQSGASNDNHYFGYSTPWGYFDFNRFHCHFSPRDWQRLINNNWGFRPKKLNFKLFNIQVKEVTQNDGTTTIANNLTSTVQVFTDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLSRTQTTSGTTQQSRLQFSQAGPSSMAQQAKNWLPGPCYRQQRVSKTANDNNNSNFAWTGATKYHLNGRDSLVNPGPAMASHKDDEEKFFPMHGVLIFGKQGTGASNVDLDNVMITDEEEIRTTNPVATEQYGTVATNLQSSNTAPATGTVNSQGALPGMVWQDRDVYLQGPIWAKIPHTDGHFHPSPLMGGFGLKHPPPQILIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 18 - DNA, 2205 bp, Adeno-associated virus
  variation 124..126: note = /replace="agt"
  variation 502..504: note = /replace="aaa"
  variation 928..930: note = /replace="aga"
  variation 1228..1230: note = /replace="cag"
  variation 1336..1338: note = /replace="aga"
  variation 1381..1383: note = /replace="ctc"
  variation 1411..1413: note = /replace="tct"
  variation 1423..1425: note = /replace="aga"
  variation 1510..1512: note = /replace="gcg"
  variation 1615..1617: note = /replace="gac"
  source 1..2205: mol_type = other DNA
atggctgctgacggttatcttccagattggctcgaggacaacctttctgaaggcattcgtgagtggtgggatctgaaacctggagcccctcaacccaaagcgaaccaacaacaccaggacgacggtcggggtcttgtgcttccgggttacaaatacctcggaccctttaacggactcgacaaaggagagccggtcaacgaggcggacgcggcagccctcgaacacgacaaagcttacgaccagcagctcaaggccggtgacaacccgtacctcaagtacaaccacgccgacgccgagtttcaggagcgtcttcaagaagatacgtcttttgggggcaaccttggcagagcagtcttccaggccaaaaagagggtccttgagcctcttggtctggttgaggaagcagctaaaacggctcctggaaagaagaggcctgtagaacagtctcctcaggaaccggactcatcatctggtattggcaaatcgggccaacagcctgccagaaaaagactaaatttcggtcagactggagactcagagtcagtcccagaccctcaacctctcggagaaccaccagcagccccctcaggtgtgggatctaatacaatggcttcaggcggtggcgcaccaatggcagacaataacgagggtgccgatggagtgggtaattcctcaggaaattggcattgcgattccacatggctgggcgacagagtcatcaccaccagcaccagaacctgggccctgcccacttacaacaaccatctctacaagcaaatctccagccaatcaggagcttcaaacgacaaccactactttggctacagcaccccttgggggtattttgactttaacagattccactgccacttctcaccacgtgactggcagcgactcattaacaacaactggggattccggcccaagaaactcaacttcaagctcttcaacatccaagttaaagaggtcacgcagaacgatggcacgacgactattgccaataaccttaccagcacggttcaagtgtttacggactcggagtatcagctcccgtacgtgctcgggtcggcgcaccaaggctgtctcccgccgtttccagcggacgtcttcatgatccctcagtatggatacctcaccctgaacaacggaagtcaagcggtgggacgctcatccttttactgcctggagtacttcccttcgcagatgctaaggactggaaataacttcacattcagctataccttcgaggatgtaccttttcacagcagctacgctcacagccagagtttggatcgcttgatgaatcctcttattgatcagtatctgtactacctgagcagaacgcaaacaacctctggaacaacccaacaatcacggctgcaatttagccaggctgggccttcgtctatggctcagcaggccaaaaattggctacctgggccctgctaccggcaacagagagtttcaaagactgctaacgacaacaacaacagtaactttgcttggacaggggccaccaaatatcatctcaatggccgcgactcgctggtgaatccaggaccagctatggccagtcacaaggacgatgaagaaaaatttttccctatgcacggcgttctaatatttggcaaacaagggacaggggcaagtaacgtagatttagataatgtaatgattacggatgaagaagagattcgtaccaccaatcctgtggcaacagagcagtatggaactgtggcaactaacttgcagagctcaaatacagctcccgcgactggaactgtcaatagtcagggggccttacctggcatggtgtggcaagatcgtgacgtgtaccttcaaggacctatctgggcaaagattcctcacacggatggacactttcatccttctcctctgatgggaggctttggactgaaacatccgcctcctcaaatcttgatcaaaaatactccggtaccggcaaatcctccgacgactttcagcccggccaagtttgcttcatttatcactcagtactccactggacaggtcagcgtggaaattgagtgggagctacagaaagaaaacagcaaacgttggaatccagagattcagtacacttccaactacaacaagtctgttaatgtggactttactgtagacactaatggtgtttatagtgaacctcgccctattggaacccggtatctcacacgaaacttg

SEQ ID NO: 19 - protein, 736 aa, Adeno-associated virus
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKKGQQPARKRLNFGQTGDSESVPDPQPLGEPPAAPSGVGSNTMAAGGGAPMADNNEGADGVGNASGNWHCDSTWLGDRVITTSTRTWALPTYNNHLYKQISSQSGGSTNDNTYFGYSTPWGYFDFNRFHCHFSPRDWQRLINNNWGFRPKRLNFKLFNIQVKEVTTNDGTTTIANNLTSTVQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQTTSGTAGNRTLQFSQAGPSSMANQAKNWLPGPCYRQQRVSKTANQNNNSNFAWTGATKYHLNGRDSLVNPGPAMATHKDDEDKFFPMSGVLIFGKQGAGNSNVDLDNVMITNEEEIKTTNPVATEQYGTVATNLQSANTAPATGTVNSQGALPGMVWQDRDVYLQGPIWAKIPHTDGHFHPSPLMGGFGLKHPPPQILIKNTPVPANPPTTFSPAKFASFITQYSTGQVSVEIEWELQKENSKRWNPEIQYTSNYNKSTNVDFAVDTNGVYSEPRPIGTRYLTRNL

SEQ ID NO: 20 - protein, 736 aa, Adeno-associated virus
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKKGQQPAKKRLNFGQTGDSESVPDPQPLGEPPAAPSGVGSNTMASGGGAPMADNNEGADGVGNASGNWHCDSTWLGDRVITTSTRTWALPTYNNHLYKQISSQSGASTNDNTYFGYSTPWGYFDFNRFHCHFSPRDWQRLINNNWGFRPKRLNFKLFNIQVKEVTTNDGTTTIANNLTSTVQVFTDSEYQLPYVLGSAHQGCLPPFPADVFMIPQYGYLTLNNGSQAVGRSSFYCLEYFPSQMLRTGNNFQFSYTFEDVPFHSSYAHSQSLDRLMNPLIDQYLYYLSRTQTTSGTAGNRELQFSQAGPSSMANQAKNWLPGPCYRQQRVSKTTNQNNNSNFAWTGATKYHLNGRDSLVNPGPAMATHKDDEDKFFPMSGVLIFGKQGAGNSNVDLDNVMITNEEEIKTTNPVATEEYGTVATNLQSANTAPATGTVNSQGALPGMVWQNRDVYLQGPIWAKIPHTDGHFHPSPLMGGFGLKHPPPQILIKNTPVPANPPTTFSPAKFASFITQYSTGQVSVEIEWELQKENSKRWNPEIQYTSNYNKSTNVDFAVDTNGVYSEPRPIGTRYLTRNL

SEQ ID NO: 21 - protein, 736 aa, Adeno-associated virus
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKKGQQPARKRLNFGQTGDSESVPDPQPLGEPPAAPSGVGSNTMAAGGGAPMADNNEGADGVGNASGNWHCDSTWLGDRVITTSTRTWALPTYNNHLYKQISSQSGGSTNDNTYFGYSTPWGYFDFNRFHCHFSPRDWQRLINNNWGFRPKRLNFKLFNIQVKEVTTNDGTTTIANNLTSTVQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQTTSGTAGNRELQFSQAGPSSMANQAKNWLPGPCYRQQRVSKTTNQNNNSNFAWTGATKYHLNGRDSLVNPGPAMATHKDDEDKFFPMSGVLIFGKQGAGNSNVDLDNVMITSEEEIKTTNPVATEEYGTVATNLQSSNTAPATGTVNSQGALPGMVWQERDVYLQGPIWAKIPHTDGHFHPSPLMGGFGLKHPPPQILIKNTPVPANPPTTFSPAKFASFITQYSTGQVSVEIEWELQKENSKRWNPEIQYTSNYNKSTNVDFAVDTNGVYSEPRPIGTRYLTRNL

SEQ ID NO: 22 - protein, 736 aa, Adeno-associated virus
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKKGQQPARKRLNFGQTGDSESVPDPQPLGEPPAAPSGVGSNTMASGGGAPMADNNEGADGVGNASGNWHCDSTWLGDRVITTSTRTWALPTYNNHLYKQISSQSGGSTNDNTYFGYSTPWGYFDFNRFHCHFSPRDWQRLINNNWGFRPKKLNFKLFNIQVKEVTTNDGTTTIANNLTSTVQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQTTSGTAGNRELQFSQAGPSSMANQAKNWLPGPCYRQQRVSKTTNQNNNSNFAWTGATKYHLNGRDSLVNPGPAMATHKDDEDKFFPMSGVLIFGKQGAGNSNVDLDNVMITSEEEIKTTNPVATEEYGTVATNLQSANTAPATGTVNSQGALPGMVWQDRDVYLQGPIWAKIPHTDGHFHPSPLMGGFGLKHPPPQILIKNTPVPANPPTTFSPAKFASFITQYSTGQVSVEIEWELQKENSKRWNPEIQYTSNYNKSTNVDFAVDTNGVYSEPRPIGTRYLTRNL

SEQ ID NO: 23 - protein, 736 aa, Adeno-associated virus
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKKGQQPARKRLNFGQTGDSESVPDPQPLGEPPAAPSGVGSNTMAAGGGAPMADNNEGADGVGNASGNWHCDSTWLGDRVITTSTRTWALPTYNNHLYKQISSQSGGSTNDNTYFGYSTPWGYFDFNRFHCHFSPRDWQRLINNNWGFRPKKLNFKLFNIQVKEVTTNDGTTTIANNLTSTVQVFTDSEYQLPYVLGSAHQGCLPPFPADVFMIPQYGYLTLNNGSQAVGRSSFYCLEYFPSQMLRTGNNFQFSYTFEDVPFHSSYAHSQSLDRLMNPLIDQYLYYLSRTQTTSGTAGNRTLQFSQAGPSSMANQAKNWLPGPCYRQQRVSKTTNQNNNSNFAWTGATKYHLNGRDSLVNPGPAMATHKDDEDKFFPMSGVLIFGKQGAGNSNVDLDNVMITNEEEIKTTNPVATEEYGTVATNLQSANTAPATGTVNSQGALPGMVWQDRDVYLQGPIWAKIPHTDGHFHPSPLMGGFGLKHPPPQILIKNTPVPANPPTTFSPAKFASFITQYSTGQVSVEIEWELQKENSKRWNPEIQYTSNYNKSTNVDFAVDTNGVYSEPRPIGTRYLTRNL

SEQ ID NO: 24 - protein, 736 aa, Adeno-associated virus
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKKGQQPAKKRLNFGQTGDSESVPDPQPLGEPPAAPSGVGSNTMAAGGGAPMADNNEGADGVGNASGNWHCDSTWLGDRVITTSTRTWALPTYNNHLYKQISSQSGGSTNDNTYFGYSTPWGYFDFNRFHCHFSPRDWQRLINNNWGFRPKKLNFKLFNIQVKEVTTNDGTTTIANNLTSTVQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQTTSGTAGNRTLQFSQAGPSSMANQAKNWLPGPCYRQQRVSKTANQNNNSNFAWTGATKYHLNGRDSLVNPGPAMATHKDDEDKFFPMSGVLIFGKQGAGNSNVDLDNVMITSEEEIKTTNPVATEQYGTVATNLQSSNTAPATGTVNSQGALPGMVWQNRDVYLQGPIWAKIPHTDGHFHPSPLMGGFGLKHPPPQILIKNTPVPANPPTTFSPAKFASFITQYSTGQVSVEIEWELQKENSKRWNPEIQYTSNYNKSTNVDFAVDTNGVYSEPRPIGTRYLTRNL

SEQ ID NO: 25 - protein, 736 aa, Adeno-associated virus
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKKGQQPAKKRLNFGQTGDSESVPDPQPLGEPPAAPSGVGSNTMASGGGAPMADNNEGADGVGNASGNWHCDSTWLGDRVITTSTRTWALPTYNNHLYKQISSQSGGSTNDNTYFGYSTPWGYFDFNRFHCHFSPRDWQRLINNNWGFRPKKLNFKLFNIQVKEVTTNDGTTTIANNLTSTVQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQTTSGTAGNRTLQFSQAGPSSMANQAKNWLPGPCYRQQRVSKTANQNNNSNFAWTGATKYHLNGRDSLVNPGPAMATHKDDEDKFFPMSGVLIFGKQGAGNSNVDLDNVMITSEEEIKTTNPVATEEYGTVATNLQSSNTAPATGTVNSQGALPGMVWQNRDVYLQGPIWAKIPHTDGHFHPSPLMGGFGLKHPPPQILIKNTPVPANPPTTFSPAKFASFITQYSTGQVSVEIEWELQKENSKRWNPEIQYTSNYNKSTNVDFAVDTNGVYSEPRPIGTRYLTRNL

SEQ ID NO: 26 - protein, 736 aa, Adeno-associated virus
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKKGQQPAKKRLNFGQTGDSESVPDPQPLGEPPAAPSGVGSNTMASGGGAPMADNNEGADGVGNASGNWHCDSTWLGDRVITTSTRTWALPTYNNHLYKQISSQSGGSTNDNTYFGYSTPWGYFDFNRFHCHFSPRDWQRLINNNWGFRPKKLNFKLFNIQVKEVTTNDGTTTIANNLTSTVQVFTDSEYQLPYVLGSAHQGCLPPFPADVFMIPQYGYLTLNNGSQAVGRSSFYCLEYFPSQMLRTGNNFQFSYTFEDVPFHSSYAHSQSLDRLMNPLIDQYLYYLSRTQTTSGTAGNRELQFSQAGPSSMANQAKNWLPGPCYRQQRVSKTTNQNNNSNFAWTGATKYHLNGRDSLVNPGPAMATHKDDEDKFFPMSGVLIFGKQGAGNSNVDLDNVMITNEEEIKTTNPVATEQYGTVATNLQSANTAPATGTVNSQGALPGMVWQDRDVYLQGPIWAKIPHTDGHFHPSPLMGGFGLKHPPPQILIKNTPVPANPPTTFSPAKFASFITQYSTGQVSVEIEWELQKENSKRWNPEIQYTSNYNKSTNVDFAVDTNGVYSEPRPIGTRYLTRNL

SEQ ID NO: 27 - DNA, 97 bp, synthetic construct
  source 1..97: mol_type = other DNA
tgctgttgacagtgagcgagcagatgagctcttagctgattagtgaagccacagatgtaatcagctaagagctcatctgcctgcctactgcctcgga

SEQ ID NO: 28 - DNA, 97 bp, synthetic construct
  source 1..97: mol_type = other DNA
tgctgttgacagtgagcgaacccaacctgtccatcattattagtgaagccacagatgtaataatgatggacaggttgggtgtgcctactgcctcgga

SEQ ID NO: 29 - DNA, 97 bp, synthetic construct
  source 1..97: mol_type = other DNA
tgctgttgacagtgagcgagctgagttcctcaaggtcaagtagtgaagccacagatgtacttgaccttgaggaactcagcctgcctactgcctcgga

SEQ ID NO: 30 - DNA, 97 bp, synthetic construct
  source 1..97: mol_type = other DNA
tgctgttgacagtgagcgctcagtcaagagcattgccaagtagtgaagccacagatgtacttggcaatgctcttgactgaatgcctactgcctcgga

SEQ ID NO: 31 - DNA, 97 bp, synthetic construct
  source 1..97: mol_type = other DNA
tgctgttgacagtgagcgacctgtctggcttctcttctactagtgaagccacagatgtagtagaagagaagccagacagggtgcctactgcctcgga

SEQ ID NO: 32 - DNA, 97 bp, synthetic construct
  source 1..97: mol_type = other DNA
tgctgttgacagtgagcgagccgagttcctcaaggtcaagtagtgaagccacagatgtacttgaccttgaggaactcggcctgcctactgcctcgga

SEQ ID NO: 33 - DNA, 97 bp, synthetic construct
  source 1..97: mol_type = other DNA
tgctgttgacagtgagcgctctccgaacgtgtatcacgtttagtgaagccacagatgtaaacgtgatacacgttcggagattgcctactgcctcgga

SEQ ID NO: 34 - DNA, 6231 bp, synthetic construct
  source 1..6231: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactaaaacaggtaagtccggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgatgcctctactaaccatgttcatgttttctttttttttctacaggtcctgggtgacgaacaggctagcgccaccatgggtaagcctatccctaaccctctcctcggtctcgattctacggccgccaccatgtctctggcagatgagctcttagctgatctcgaagaggcagcagaagaggaggaaggaggaagctatggggaggaagaagaggagccagcgatcgaggatgtgcaggaggagacacagctggatctttccggggattcagtcaagaccatcgccaagctatgggatagtaagatgtttgctgagattatgatgaagattgaggagtatatcagcaagcaagccaaagcttcagaagtgatgggaccagtggaggccgcgcctgaataccgcgtcatcgtggatgccaacaacctgaccgtggagatcgaaaacgagctgaacatcatccataagttcatccgggataagtactcaaagagattccctgaactggagtccttggtccccaatgcactggattacatccgcacggtcaaggagctgggcaacagcctggacaagtgcaagaacaatgagaacctgcagcagatcctcaccaatgccaccatcatggtcgtcagcgtcaccgcctccaccacccaggggcagcagctgtcggaggaggagctggagcggctggaggaggcctgcgacatggcgctggagctgaacgcctccaagcaccgcatctacgagtatgtggagtcccggatgtccttcatcgcacccaacctgtccatcattatcggggcatccacggccgccaagatcatgggtgtggccggcggcctgaccaacctctccaagatgcccgcctgcaacatcatgctgctcggggcccagcgcaagacgctgtcgggcttctcgtctacctcagtgctgccccacaccggctacatctaccacagtgacatcgtgcagtccctgccaccggatctgcggcggaaagcggcccggctggtggccgccaagtgcacactggcagcccgtgtggacagtttccacgagagcacagaagggaaggtgggctacgaactgaaggatgagatcgagcgcaaattcgacaagtggcaggagccgccgcctgtgaagcaggtgaagccgctgcctgcgcccctggatggacagcggaagaagcgaggcggccgcaggtaccgcaagatgaaggagcggctggggctgacggagatccggaagcaggccaaccgtatgagcttcggagagatcgaggaggacgcctaccaggaggacctgggattcagcctgggccacctgggcaagtcgggcagtgggcgtgtgcggcagacacaggtaaacgaggccaccaaggccaggatctccaagacgctgcagcggaccctgcagaagcagagcgtcgtatatggcgggaagtccaccatccgcgaccgctcctcgggcacggcctccagcgtggccttcaccccactccagggcctggagattgtgaacccacaggcggcagagaagaaggtggctgaggccaaccagaagtatttctccagcatggctgagttcctcaaggtcaagggcgagaagagtggccttatgtccacctgaaccggttggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcggatccgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaatagtggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagtaatggtaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcggttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 35 - DNA, 108 bp, synthetic construct
  source 1..108: mol_type = other DNA
caccgcatctacgagtatgtggagtcccggatgtccttcatcgcacccaacctgtccatcattatcggggcatccacggccgccaagatcatgggtgtggccggcggc

SEQ ID NO: 36 - DNA, 108 bp, synthetic construct
  source 1..108: mol_type = other DNA
caccgcatctacgagtggagtcccggatgtccttcatcgcacccaacctgtccatcattatcggggcatccacggccgccaacatcatgggtgtggccggcggcctga

SEQ ID NO: 37 - skipped

SEQ ID NO: 38 - DNA, 108 bp, synthetic construct
  source 1..108: mol_type = other DNA
caccgcatctacatgtggagtcccggatgtccttcatcgcacccaacctgtccatcattatcggggcatccacggccgccaacatcatgggtgtggccggcggcctga

SEQ ID NO: 39 - DNA, 105 bp, synthetic construct
  source 1..105: mol_type = other DNA
caccgcatctacgagtatgtggagtcccggatgtccttcatcgcacccaacctgtccatcattatcggggcatccacggccgccaagatcatgggtcacagtgac

SEQ ID NO: 40 - DNA, 105 bp, synthetic construct
  source 1..105: mol_type = other DNA
caccgcatctacgagttatgtggagtcccggatgtccttcatcgcacccaacctgtccatcattatcggggcatccacggccgccaagatcatgggccacagtga

SEQ ID NO: 41 - protein, 36 aa, synthetic construct
HRIYEYVESRMSFIAPNLSIIIGASTAAKIMGVAGG

SEQ ID NO: 42 - protein, 35 aa, synthetic construct
HRIYEWSPGCPSSHPTCPSLSHHPRPPRSWVWPAA

SEQ ID NO: 43 - protein, 35 aa, synthetic construct
HRIYMWSPGCPSSHPTCPSLSHHPRPPRSWVWPAA

SEQ ID NO: 44 - protein, 35 aa, synthetic construct
HRIYEYVESRMSFIAPNLSIIIGASTAAKIMGHSD

SEQ ID NO: 45 - protein, 34 aa, synthetic construct
HRIYELCGVPDVLHRTQPVHHYRGIHGRQDHGPQ